SEQUENCE LISTING (28 sequences)

SEQ ID NO: 1 - DNA, 33 bp, synthetic construct
  source 1..33: mol_type = other DNA; note = SA1-Propynyl
  modified_base 2: mod_base = OTHER; note = Propynyl-dC modification
  modified_base 4: mod_base = OTHER; note = Propynyl-dC modification
  modified_base 6: mod_base = OTHER; note = Propynyl-dC modification
  modified_base 10..12: mod_base = OTHER; note = Propynyl-dC modification
  modified_base 15: mod_base = OTHER; note = Propynyl-dC modification
  modified_base 17: mod_base = OTHER; note = Propynyl-dC modification
  modified_base 20: mod_base = OTHER; note = Propynyl-dC modification
  modified_base 22: mod_base = OTHER; note = Propynyl-dC modification
  modified_base 24: mod_base = OTHER; note = Propynyl-dC modification
  modified_base 27..28: mod_base = OTHER; note = Propynyl-dC modification
  modified_base 32: mod_base = OTHER; note = Propynyl-dC modification
  misc_feature 32..33: note = phosphorothioate bond
acactctttccctacacgacgctcttccgatct

SEQ ID NO: 2 - DNA, 31 bp, synthetic construct
  source 1..31: mol_type = other DNA; note = SA2-propynyl
  modified_base 4: mod_base = OTHER; note = Propynyl-dC modification
  modified_base 12: mod_base = OTHER; note = Propynyl-dC modification
  modified_base 14: mod_base = OTHER; note = Propynyl-dC modification
  modified_base 16: mod_base = OTHER; note = Propynyl-dC modification
  modified_base 19: mod_base = OTHER; note = Propynyl-dC modification
  modified_base 24: mod_base = OTHER; note = Propynyl-dC modification
  modified_base 26..27: mod_base = OTHER; note = Propynyl-dC modification
  modified_base 31: mod_base = OTHER; note = Propynyl-dC modification
gatcggaagagcacacgtctgaactccagtc

SEQ ID NO: 3 - DNA, 33 bp, synthetic construct; identical to SEQ ID NO: 1
  source 1..33: mol_type = other DNA
  modified_base 2: mod_base = OTHER; note = 5-methyl-dC modification
  modified_base 4: mod_base = OTHER; note = 5-methyl-dC modification
  modified_base 6: mod_base = OTHER; note = 5-methyl-dC modification
  modified_base 10..12: mod_base = OTHER; note = 5-methyl-dC modification
  modified_base 15: mod_base = OTHER; note = 5-methyl-dC modification
  modified_base 17: mod_base = OTHER; note = 5-methyl-dC modification
  modified_base 20: mod_base = OTHER; note = 5-methyl-dC modification
  modified_base 22: mod_base = OTHER; note = 5-methyl-dC modification
  modified_base 24: mod_base = OTHER; note = 5-methyl-dC modification
  modified_base 27..28: mod_base = OTHER; note = 5-methyl-dC modification
  modified_base 32: mod_base = OTHER; note = 5-methyl-dC modification
  misc_feature 32..33: note = phosphorothioate bond

SEQ ID NO: 4 - DNA, 31 bp, synthetic construct; identical to SEQ ID NO: 2
  source 1..31: mol_type = other DNA; note = SA2-Methyl
  modified_base 4: mod_base = OTHER; note = 5-methyl-dC modification
  modified_base 12: mod_base = OTHER; note = 5-methyl-dC modification
  modified_base 14: mod_base = OTHER; note = 5-methyl-dC modification
  modified_base 16: mod_base = OTHER; note = 5-methyl-dC modification
  modified_base 19: mod_base = OTHER; note = 5-methyl-dC modification
  modified_base 24: mod_base = OTHER; note = 5-methyl-dC modification
  modified_base 26..27: mod_base = OTHER; note = 5-methyl-dC modification
  modified_base 31: mod_base = OTHER; note = 5-methyl-dC modification

SEQ ID NO: 5 - DNA, 33 bp, synthetic construct; identical to SEQ ID NO: 1
  source 1..33: mol_type = other DNA; note = SA1-pyrrolo
  modified_base 2: mod_base = OTHER; note = pyrrolo-dC modification
  modified_base 4: mod_base = OTHER; note = pyrrolo-dC modification
  modified_base 6: mod_base = OTHER; note = pyrrolo-dC modification
  modified_base 10..12: mod_base = OTHER; note = pyrrolo-dC modification
  modified_base 15: mod_base = OTHER; note = pyrrolo-dC modification
  modified_base 17: mod_base = OTHER; note = pyrrolo-dC modification
  modified_base 20: mod_base = OTHER; note = pyrrolo-dC modification
  modified_base 22: mod_base = OTHER; note = pyrrolo-dC modification
  modified_base 24: mod_base = OTHER; note = pyrrolo-dC modification
  modified_base 27..28: mod_base = OTHER; note = pyrrolo-dC modification
  modified_base 32: mod_base = OTHER; note = pyrrolo-dC modification
  misc_feature 32..33: note = phosphorothioate bond

SEQ ID NO: 6 - DNA, 31 bp, synthetic construct; identical to SEQ ID NO: 2
  source 1..31: mol_type = other DNA; note = SA2-pyrrolo
  modified_base 4: mod_base = OTHER; note = Pyrrolo-dC modification
  modified_base 12: mod_base = OTHER; note = Pyrrolo-dC modification
  modified_base 14: mod_base = OTHER; note = Pyrrolo-dC modification
  modified_base 16: mod_base = OTHER; note = Pyrrolo-dC modification
  modified_base 19: mod_base = OTHER; note = Pyrrolo-dC modification
  modified_base 24: mod_base = OTHER; note = Pyrrolo-dC modification
  modified_base 26..27: mod_base = OTHER; note = Pyrrolo-dC modification
  modified_base 31: mod_base = OTHER; note = Pyrrolo-dC modification

SEQ ID NO: 7 - DNA, 30 bp, synthetic construct
  source 1..30: mol_type = other DNA; note = SA-5hmC
  modified_base 4: mod_base = OTHER; note = 5hmC modification
  modified_base 12: mod_base = OTHER; note = 5hmC modification
  modified_base 14: mod_base = OTHER; note = 5hmC modification
  modified_base 16: mod_base = OTHER; note = 5hmC modification
  modified_base 19: mod_base = OTHER; note = 5hmC modification
  modified_base 24: mod_base = OTHER; note = 5hmC modification
  modified_base 26..27: mod_base = OTHER; note = 5hmC modification
gatcggaagagcacacgtctgaactccagt

SEQ ID NO: 8 - DNA, 33 bp, synthetic construct; identical to SEQ ID NO: 1
  source 1..33: mol_type = other DNA; note = SA2-5hmC
  modified_base 2: mod_base = OTHER; note = 5hmC modification
  modified_base 4: mod_base = OTHER; note = 5hmC modification
  modified_base 6: mod_base = OTHER; note = 5hmC modification
  modified_base 10..12: mod_base = OTHER; note = 5hmC modification
  modified_base 15: mod_base = OTHER; note = 5hmC modification
  modified_base 17: mod_base = OTHER; note = 5hmC modification
  modified_base 20: mod_base = OTHER; note = 5hmC modification
  modified_base 22: mod_base = OTHER; note = 5hmC modification
  modified_base 24: mod_base = OTHER; note = 5hmC modification
  modified_base 27..28: mod_base = OTHER; note = 5hmC modification
  modified_base 32: mod_base = OTHER; note = 5hmC modification
  misc_feature 32..33: note = phosphorothioate bond

SEQ ID NO: 9 - DNA, 272 bp, synthetic construct
  source 1..272: mol_type = other DNA; note = Oligonucleotide
gtcactcagatgtatagaatgatgagttaggtagtgttgatatgggttatgaatgaagtagtcgatctttcatcatattctagatccctctgaaaaaatcttccgagtttgctaggcagtgatacataactcttttccaataattggggaagtcattcaaatctataataggtttcagatttaattctgactgtagctgctgaaacgttgcggagtgttaaggtatatgagtagatgattgattgggtatgttgataagtgtagtcactcag

SEQ ID NO: 10 - DNA, 51 bp, synthetic construct
  source 1..51: mol_type = other DNA; note = OTF12
atgtatagaatgatgagttaggtagtgttgatatgggttatgaatgaagta

SEQ ID NO: 11 - DNA, 50 bp, synthetic construct
  source 1..50: mol_type = other DNA; note = OTR12
tacacttatcaacatacccaatcaatcatctactcatataccttaacact

SEQ ID NO: 12 - DNA, 57 bp, synthetic construct
  source 1..57: mol_type = other DNA; note = OTF2
acactctttccctacacgacgctcttccgatctttgatatgggttatgaatgaagta

SEQ ID NO: 13 - DNA, 57 bp, synthetic construct
  source 1..57: mol_type = other DNA; note = OTR2
gactggagttcagacgtgtgctcttccgatctagtgttaaggtatatgagtagatga

SEQ ID NO: 14 - DNA, 163 bp, synthetic construct
  source 1..163: mol_type = other DNA; note = Oligonucleotide
atatagtgtgtaatattaagggagaattggctgctgccgctaaagatagtttagatatggaatgacccgggacgatacgtattcaaaggtatcatgaaacgttggtcataatagatgattgagatttaagtatttgttgagttgatgttgtttattggcgcgc

SEQ ID NO: 15 - DNA, 88 bp, synthetic construct
  source 1..88: mol_type = other DNA; note = Spike In F
  modified_base 68: mod_base = OTHER; note = 5-hydroxymethyl dC modification
  modified_base 78: mod_base = OTHER; note = 5-Methyl dC modification
  modified_base 84: mod_base = OTHER; note = 5-Methyl dC modification
atatagtgtgtaatattaagggagaattggctgctgccgctaaagatagtttagatatggaatgacccgggacgatacgtattcaaag

SEQ ID NO: 16 - DNA, 33 bp, synthetic construct
  source 1..33: mol_type = other DNA; note = Spike_in_R
gcgcgccaataaacaacatcaactcaacaaata

SEQ ID NO: 17 - DNA, 24 bp, synthetic construct
  source 1..24: mol_type = other DNA; note = Post deamination primer
gtgtgtaatattaagggagaattg

SEQ ID NO: 18 - DNA, 26 bp, synthetic construct
  source 1..26: mol_type = other DNA; note = Post deamination primer
aataaacaacatcaactcaacaaata

SEQ ID NO: 19 - DNA, 57 bp, synthetic construct
  source 1..57: mol_type = other DNA; note = primer
acactctttccctacacgacgctcttccgatctgtgtgtaatattaagggagaattg

SEQ ID NO: 20 - DNA, 58 bp, synthetic construct
  source 1..58: mol_type = other DNA; note = primer
gactggagttcagacgtgtgctcttccgatctaataaacaacatcaactcaacaaata

SEQ ID NO: 21 - DNA, 62 bp, synthetic construct
  source 1..62: mol_type = other DNA; note = Representative Candidate Adapter
  modified_base 8: mod_base = OTHER; note = Variant Cytosine
  modified_base 11: mod_base = OTHER; note = Variant Cytosine
  modified_base 14..15: mod_base = OTHER; note = Variant Cytosine
  modified_base 17..18: mod_base = OTHER; note = Variant Cytosine
  modified_base 24: mod_base = OTHER; note = Variant Cytosine
  modified_base 27: mod_base = OTHER; note = Variant Cytosine
  modified_base 29: mod_base = OTHER; note = Variant Cytosine [i5]
  modified_base 31: mod_base = OTHER; note = Variant Cytosine
  modified_base 33: mod_base = OTHER; note = Variant Cytosine
  modified_base 35: mod_base = OTHER; note = Variant Cytosine
  modified_base 39..41: mod_base = OTHER; note = Variant Cytosine
  modified_base 44: mod_base = OTHER; note = Variant Cytosine
  modified_base 46: mod_base = OTHER; note = Variant Cytosine
  modified_base 49: mod_base = OTHER; note = Variant Cytosine
  modified_base 51: mod_base = OTHER; note = Variant Cytosine
  modified_base 53: mod_base = OTHER; note = Variant Cytosine
  modified_base 56..57: mod_base = OTHER; note = Variant Cytosine
  modified_base 61: mod_base = OTHER; note = Variant Cytosine
  misc_feature 61..62: note = phosphorothioate bond
aatgatacggcgaccaccgagatctacacacactctttccctacacgacgctcttccgatct

SEQ ID NO: 22 - DNA, 57 bp, synthetic construct
  source 1..57: mol_type = other DNA; note = Representative Candidate Adapter
  modified_base 4: mod_base = OTHER; note = Variant Cytosine
  modified_base 12: mod_base = OTHER; note = Variant Cytosine
  modified_base 14: mod_base = OTHER; note = Variant Cytosine
  modified_base 16: mod_base = OTHER; note = Variant Cytosine
  modified_base 19: mod_base = OTHER; note = Variant Cytosine
  modified_base 24: mod_base = OTHER; note = Variant Cytosine
  modified_base 26..27: mod_base = OTHER; note = Variant Cytosine
  modified_base 31: mod_base = OTHER; note = Variant Cytosine
  modified_base 33: mod_base = OTHER; note = Variant Cytosine [i7]
  modified_base 36: mod_base = OTHER; note = Variant Cytosine
  modified_base 38: mod_base = OTHER; note = Variant Cytosine
  modified_base 44..45: mod_base = OTHER; note = Variant Cytosine
  modified_base 48: mod_base = OTHER; note = Variant Cytosine
  modified_base 51: mod_base = OTHER; note = Variant Cytosine
  modified_base 54: mod_base = OTHER; note = Variant Cytosine
gatcggaagagcacacgtctgaactccagtcacatctcgtatgccgtcttctgcttg

SEQ ID NO: 23 - DNA, 33 bp, synthetic construct; identical to SEQ ID NO: 1
  source 1..33: mol_type = other DNA; note = Representative Candidate Adapter
  misc_feature 2: note = Variant Cytosine
  misc_feature 4: note = Variant Cytosine
  misc_feature 6: note = Variant Cytosine
  misc_feature 10..12: note = Variant Cytosine
  misc_feature 15: note = Variant Cytosine
  misc_feature 17: note = Variant Cytosine
  misc_feature 17: note = Variant Cytosine
  misc_feature 20: note = Variant Cytosine
  misc_feature 22: note = Variant Cytosine
  misc_feature 24: note = Variant Cytosine
  misc_feature 27..28: note = Variant Cytosine
  misc_feature 32: note = Variant Cytosine
  misc_feature 32..33: note = phosphorothioate bond

SEQ ID NO: 24 - DNA, 31 bp, synthetic construct; identical to SEQ ID NO: 2
  source 1..31: mol_type = other DNA; note = Representative Candidate Adapter
  misc_feature 4: note = Variant Cytosine
  misc_feature 12: note = Variant Cytosine
  misc_feature 14: note = Variant Cytosine
  misc_feature 16: note = Variant Cytosine
  misc_feature 19: note = Variant Cytosine
  misc_feature 24: note = Variant Cytosine
  misc_feature 26..27: note = Variant Cytosine
  misc_feature 31: note = Variant Cytosine

SEQ ID NO: 25 - DNA, 65 bp, synthetic construct
  source 1..65: mol_type = other DNA; note = Representative Candidate Adapter
  misc_feature 4: note = Variant Cytosine
  misc_feature 12: note = Variant Cytosine
  misc_feature 14: note = Variant Cytosine
  misc_feature 19: note = Variant Cytosine
  misc_feature 24: note = Variant Cytosine
  misc_feature 26..27: note = Variant Cytosine
  misc_feature 31: note = Variant Cytosine
  misc_feature 32: note = Uracil
  misc_feature 34: note = Variant Cytosine
  misc_feature 36: note = Variant Cytosine
  misc_feature 38: note = Variant Cytosine
  misc_feature 42..44: note = Variant Cytosine
  misc_feature 47: note = Variant Cytosine
  misc_feature 49: note = Variant Cytosine
  misc_feature 52: note = Variant Cytosine
  misc_feature 54: note = Variant Cytosine
  misc_feature 56: note = Variant Cytosine
  misc_feature 59..60: note = Variant Cytosine
  misc_feature 64: note = Variant Cytosine
  misc_feature 64..65: note = Phosphorothioate bond
gatcggaagagcacacgtctgaactccagtctacactctttccctacacgacgctcttccgatct

SEQ ID NO: 26 - DNA, 55 bp, synthetic construct
  source 1..55: mol_type = other DNA; note = Hairpin linker
  modified_base 1: mod_base = OTHER; note = Variant Cytosine
  modified_base 4..5: mod_base = OTHER; note = Variant Cytosine
  modified_base 12: mod_base = OTHER; note = Variant Cytosine
  variation 17..21: note = a or c or g or t
  variation 22..37: note = a or c or g or t or absent
  modified_base 41..42: mod_base = OTHER; note = Variant Cytosine
  modified_base 43..44: mod_base = OTHER; note = Variant Cytosine
  misc_feature 17..37: note = barcode
  modified_base 46..47: mod_base = OTHER; note = Variant Cytosine
  misc_feature 51: note = Uracil
  misc_feature 53..54: note = Phosphorothioate bond
cgaccaggtggcaagtgnnnnnnnnnnnnnnnnnnnnaagccaccacctggtcgt

SEQ ID NO: 27 - DNA, 30 bp, synthetic construct
  source 1..30: mol_type = other DNA
gatttaattctgactgtagctgctgaaacg

SEQ ID NO: 28 - DNA, 30 bp, synthetic construct
  source 1..30: mol_type = other DNA
gatttaattttgattgtagttgttgaaatg

(19) United States
(12) Patent Application Publication
(10) Pub. No.: US 2025/0283170 A1
(43) Pub. Date: Sep. 11, 2025

(54) MODIFIED ADAPTERS FOR ENZYMATIC DNA DEAMINATION AND METHODS OF USE THEREOF FOR EPIGENETIC SEQUENCING OF FREE AND IMMOBILIZED DNA

(71) Applicant: THE TRUSTEES OF THE UNIVERSITY OF PENNSYLVANIA, Philadelphia, PA (US)

(72) Inventors: Rahul KOHLI, Penn Valley, PA (US); Tong WANG, Philadelphia, PA (US); Christian LOO, Philadelphia, PA (US)

(73) Assignee: THE TRUSTEES OF THE UNIVERSITY OF PENNSYLVANIA, Philadelphia, PA (US)

(21) Appl. No.: 18/578,997

(22) PCT Filed: Jul. 12, 2022
(86) PCT No.: PCT/US2022/073643

§ 371 Date: Jan. 12, 2024

Related U.S. Application Data
(60) Provisional application No. 63/220,650, filed on Jul. 12, 2021.

(51) Int. Cl. C12Q 1/6876; C12Q 1/6806; C12Q 1/6874

(52) U.S. Cl. CPC C12Q 1/6876; C12Q 1/6806; C12Q 1/6874; C12Q 2600/154

(57) ABSTRACT

Compositions and methods for profiling methylation patterns present on target DNA in solution or affixed to a solid support are disclosed using enzymatic deamination-resistant and optionally also chemically resistant, oligonucleotides and nucleotides.

### CROSS REFERENCE TO RELATED APPLICATION

This application is a § 371 of International Application No. PCT/US2022/073643, filed Jul. 12, 2022, which claims priority to U.S. Provisional Application No. 63/220,650, filed on Jul. 12, 2021, the entire contents of each being incorporated herein by reference as though set forth in full.

### GRANT STATEMENT

This invention was made with government support under HG010646 awarded by the National Institutes of Health. The government has certain rights in the invention.

### REFERENCE TO AN ELECTRONIC SEQUENCE LISTING

The contents of the electronic sequence listing (UPNK-109-US01.xml; 114,696 bytes; and Date of Creation: Aug. 20, 2024) is herein incorporated by reference in its entirety.

### FIELD OF THE INVENTION

This invention relates the fields of epigenetics and means for efficient analysis of modifications to cytosine bases present in genomic DNA target sequences using modified adapters or nucleotides with cytosine analogs that are resistant to enzymatic deamination and applying these to the profiling of free DNA or DNA immobilized on solid supports using the modified adapters.

### BACKGROUND OF THE INVENTION

Several publications and patent documents are cited throughout the specification in order to describe the state of the art to which this invention pertains. Each of these citations is incorporated herein by reference as though set forth in full.

The four chemically distinct bases of DNA—A, C, G, and T—are conserved across phylogeny and provide genomic material which can be inherited across generations. Early in the 20^{th} century, however, Wheeler and Johnson first synthesized 5-methylcytosine (5mC) and postulated about its existence in genomic DNA samples. Presciently called ‘epicytosine’ in later studies by Hotchkiss, 5mC was shown to have a distinct chemical identity from its parent base while maintaining many of its same properties [1,2].

Several decades later, the ubiquity of 5mC became evident, solidifying its standing as the 5^{th} base of genomic DNA. From prokaryotes to eukaryotes, a conserved family of DNA methyltransferase enzymes (MTases) has been shown to catalyze the generation of 5mC through reaction between the unmodified cytosine in DNA and the methyl donor S-adenosyl-L-methionine (SAM). 5mC preserves the hydrogen bonding capacity for pairing with guanine that is required for successful DNA replication. However, the methyl moiety introduced at the 5-position of cytosine provides a readable chemical handle that has the potential to affect DNA-binding proteins and enzymes which often interact within the major groove of DNA, thus implicating 5mC across many diverse processes. In bacterial species, this chemical mark can serve to distinguish self from non-self as part of restriction-modification systems [3]. In eukaryotes, 5mC takes on new functions, serving predominantly as a gene repressive epigenetic marker with physiological roles in development, imprinting, X-chromosome inactivation, and transposon silencing, as well as pathological roles in oncogenesis [4]. In 5mC, nature has found an opportunity to embellish DNA, thus expanding its information-encoding capacity within each generation without affecting DNA's most important function for inheritance of information across generations [5].

While early approaches such as paper chromatography and restriction digestion provided a means for distinguishing 5mC from its parent base [2,6], it was the subsequent application of the chemical sodium bisulfite (NaHSO₃) that allowed for the study of methylated cytosines at base resolution (FIG. 1A). The treatment of genomic DNA with bisulfite (BS) under acidic conditions leads to the sulfonation of unmodified cytosines, which promotes their deamination to uracil [7]. By contrast, 5mC does not react efficiently with bisulfite. Following amplification, the unmodified cytosines are read as thymidine in sequencing, while 5mC is still read as cytosine.

The last decade has expanded our understanding of the importance of modified cytosines in epigenetics even further [4,5]. The discovery of the TET family of enzymes [8] demonstrated that 5mC could be oxidized as part of a pathway promoting the reversion of 5mC back to unmodified cytosine, a pathway known as active DNA demethylation. TET dioxygenases catalyze the stepwise conversion of 5mC to 5-hydroxymethyl (5hmC) (FIG. 1B), 5hmC to 5-formyl (5fC), and 5fC to 5-carboxylcytosine (5caC) [9,10]. 5hmC is the most prevalent of these modifications, reaching as much as 10-30% of the level of 5mC in certain contexts like in cerebellar Purkinje cells [11]. Importantly, the field's reliance on bisulfite in part explains why 5hmC was long overlooked (FIG. 1A). Unlike 5mC, 5hmC reacts with bisulfite, generating cytosine-5-methylenesulfonate (CMS). However, as CMS base pairs with G upon amplification, the initial 5hmC base is indistinguishable from 5mC upon sequencing [12].

Clearly, there is a need in the art to improve the efficiency and accuracy of cytosine methylation profiling in order to more fully characterize these epigenetic changes that affect gene expression and function.

### SUMMARY OF THE INVENTION

In accordance with the present invention, an oligonucleotide comprising an adapter harboring a modified cytosine base including without limitation, 5-propynyl-dC (5pyC), 5-pyrrolo-dC (5pyrC), 5hmC along with modified variants thereof, cytosine 5-methylenesulfonate (CMS), glucosylated 5hmC (5ghmC), bulky 5-position adducts and N4-modified base analogs which confer resistance to enzymatic deamination, chemical deamination, or both is provided. In certain embodiments, the adapter is at both ends of a DNA sample of interest and can further comprise an optional barcode sequence at one or both ends of the oligonucleotide. In preferred embodiments, the modification is 5pyC, 5pyrC, 5hmC or variants thereof. In certain embodiments, the oligonucleotides described above can be operably linked to a first member of a specific binding pair. Preferred binding pair members, include, without limitation, streptavidin-biotin, avidin-biotin, biotin analog-avidin, desthiobiotin-streptavidin, desthiobiotin-avidin, iminobiotin-streptavidin, iminobiotin-avidin, antigen-antibody, receptor-hormone, receptor-ligand, agonist-antagonist, lectin-carbohydrate, nucleic acid (RNA or DNA) hybridizing sequences, Fc receptor or mouse IgG-protein A, and virus-receptor interactions. In certain embodiments the first specific binding pair member is biotin. When the first specific binding pair member is biotin, the second specific binding pair member can be avidin or streptavidin, said second specific binding pair member being operably linked to a solid support, for example, a magnetic particle or bead. In solution-based epigenetic sequencing, a binding pair is not present.

Also provided is a method for identifying cytosine modification states in an immobilized target DNA molecule. An exemplary method comprises providing a nucleic acid sample comprising methylated DNA (which is defined as encompassing DNA containing any mixture of methylation (5mC), hydroxymethylation (5hmC), or additional natural modifications of 5mC), ligating an oligonucleotide comprising at least a first member of a specific binding pair and an adapter as described above to the modified DNA and contacting the ligated DNA with a bead or particle comprising the second member of said specific binding pair, thereby forming a duplex DNA containing specific binding member pair complex on a surface of said solid-phase (known as a bead, particle or resin). The duplex DNA tethered to the solid phase by the binding pair complex is then incubated under conditions which denature said duplex DNA, thereby producing single-stranded DNA. The single-stranded DNA is treated with at least one deaminase and PCR amplified followed by sequencing of PCR amplicons and generation of methylation profiles for the target DNA molecule. In certain embodiments, the methylated DNA is treated with at least one glucosyltransferase, methyltransferase, polymerases, and/or TET enzyme, and the appropriate substrate therefor, with these treatments taking place before or after immobilization on the solid-phase and denaturation. In other embodiments, the methylated DNA is sheared or is naturally between 50 to 1000, between 50 to 800, between 50 to 600, between 50 to 400, and between 50 to 200 nucleotides in length.

In certain embodiments, the conjugation of the modified DNA to the adapter sequence is performed using an alternative tagging strategy, e.g., a transposon, rather than through conventional DNA ligation.

In certain aspects, methylated DNA is contacted with a glucosyltransferase and UDP glucose or a chemically-modified UDP glucose derivative containing an azide functional group, thereby site-specifically labeling all 5hmC bases prior to performance of subsequent steps.

In other embodiments, the methylated DNA is contacted with at least one TET enzyme thereby catalyzing oxidation of 5mC to 5hmC, 5hmC to 5fC and 5fC to 5caC prior to performance of subsequent steps. When performed concurrently with a glucosyltransferase, the coupled action can result in the conversion of 5mC to 5ghmC.

In another approach, the methylated DNA is contacted with a methyltransferase or methyltransferase variant, thereby converting unmodified CpGs into 5-modified-CpGs. In other embodiments this methyltransferase variant is an engineered DNA carboxymethyltransferase (CxMTase) which uses carboxy-SAM (CxSAM) to convert unmodified cytosines to 5-carboxymethylcytosines.[13].

In certain aspects, the methylated DNA is copied with either deamination-resistant or non-resistant cytosine analogs to generate a homogeneously modified copy strand of the target strand. In certain embodiments, these deaminase-resistant cytosine analogs include the modifications that are shown herein to be resistant to DNA deaminases, including without limitation, 5-propynyl-dC (5pyC), 5-pyrrolo-dC (5pyrC), 5hmC along with modified variants thereof, cytosine 5-methylenesulfonate (CMS), glucosylated 5hmC (5ghmC), bulky 5-position adducts and N4-modified base analogs.

In another aspect of the invention, a method is provided for the interrogation of both genetic and epigenetic information from methylated DNA. An exemplary method entails generating a copy of the input DNA strand which is generated containing deamination-resistant cytosine analogs. In certain embodiments, this copy strand is tethered to the original strand by a linker oligonucleotide. In some embodiments, the molecule containing the linked target strand and deamination-resistant copy strand are also linked to sequencing adapters that are resistant to enzymatic deamination. The sample is then treated with at least one deaminase and PCR amplified followed by sequencing of PCR amplicons and generation of methylation profiles and original genetic profiles for the target DNA molecules. In certain embodiments, the methylated DNA is treated with at least one glucosyltransferase, methyltransferase, and TET enzyme, and the appropriate substrate therefor, with these treatments taking place before or after immobilization on the solid-phase and/or denaturation. In other embodiments, the methylated DNA is sheared or is naturally between 50 to 1000, between 50 to 800, between 50 to 600, between 50 to 400, and between 50 to 200 nucleotides in length.

In other aspects, the oligonucleotide linker contains deamination-resistant modified cytosines and an optional barcode.

In certain aspects, the DNA generated with modified cytosines is contacted with a biotinylated probe spanning a genomic region of interest post sequencing library preparation allowing for the enrichment of certain genomic loci.

In other embodiments, the methylated DNA is contacted with at least one TET enzyme thereby catalyzing oxidation of 5mC to 5hmC, 5hmC to 5fC and 5fC to 5caC prior to performance of subsequent steps. When performed concurrently with a glucosyltransferase, the coupled action can result in the conversion of 5mC to 5ghmC.

In another approach, the methylated DNA is contacted with a methyltransferase or methyltransferase variant, thereby converting unmodified CpGs into 5-modified-CpGs. In other embodiments this methyltransferase variant is an engineered DNA carboxymethyltransferase (CxMTase) which uses carboxy-SAM (CxSAM) to convert unmodified cytosines to 5-carboxymethylcytosines.

In yet another aspect of the invention, a method for reiterative assessment of the methylation state of the same DNA molecule in a plurality of library constructs is disclosed. An exemplary method entails providing a nucleic acid sample comprising methylated DNA, ligating an oligonucleotide comprising at least a first member of a specific binding pair and an adapter as described above to the methylated DNA and contacting the ligated DNA with a solid phase (referred to as a bead, particle, or resin), comprising the second member of said specific binding pair, thereby forming a duplex DNA containing specific binding member pair complex on a surface of said bead or particle. The duplex DNA containing specific binding pair complex is converted with bisulfite, thereby converting cytosine to uracil, and converting 5hmC to adduct CMS. The bisulfite-treated DNA is amplified and sequenced thereby creating a first library of constructs comprising a first set of barcoded samples, for identifying 5mC and 5hmC present in said sequence.

Subsequently, after removal of the PCR product, the DNA containing the specific binding pair complex is incubated with at least one deaminase, thereby converting 5mC to T. The immobilized DNA is then treated with bisulfite and the deaminated DNA is amplified with a distinctive barcode that thereby creating a second library for distinguishing 5mC (which was deaminated) from 5hmC (which remained resistant to deamination) present in said sequence. The first and second sets of barcodes present in the first and second library constructs are then compared, and 5mC and 5hmC modifications present in the original starting methylated DNA can be identified. In certain embodiments, the identification of molecules amplified in both libraries can be carried out by using the distinctive 5′- and 3′-ends of the molecules, rather than using a barcode encoded on the adapter molecule itself. In certain aspects of this method, the methylated DNA of step a) is treated with at least one glucosyltransferase, methyltransferase, polymerase, and TET enzyme, and the appropriate substrate therefor.

In other embodiments, the methylated sample DNA is copied by a polymerase with cytosine analogs resistant to chemical and/or enzymatic deamination and the copy strand is tethered to the original strand. This tethered molecule is then ligated to an oligonucleotide comprising at least a first member of a specific binding pair and an adapter as described above to the methylated DNA and contacting the ligated DNA with a bead or particle comprising the second member of said specific binding pair, thereby forming a duplex DNA containing specific binding member pair complex on a surface of said bead or particle. This molecule is then subjected to the above treatments enabling for the state of C, 5mC, and 5hmC to be determined while maintaining the original genetic code.

In certain embodiments, the methylated DNA is obtained from a cultured cell, a tumor cell, plasma, serum, aspirate, a swab, or a nasal secretion. In other embodiments the methylated DNA can be obtained from tissue, blood, urine, effusion, CSF, lavage, breast milk, synovial fluid, saliva, sputum, tears, abscess. In other embodiments, the methylated DNA is circulating cell-free DNA (cfDNA) present in serum or plasma. In other aspects, cfDNA can be from diseased tissue or can be of fetal origin in maternal circulation.

Kits comprising reagents and components useful for practicing the methods described above are also within the scope of the invention, along with instruments that use the methods or kits for application of the methods to immobilized DNA.

### BRIEF DESCRIPTION OF THE DRAWINGS

FIGS. 1A-1B: Bisulfite sequencing and its limitations. FIG. 1A) Bisulfite leads to selective deamination of various cytosine modifications, which can aid in localizing modifications upon PCR amplification and sequencing. Problematically, sodium bisulfite is both destructive and unable to distinguish between the two most common modifications in mammalian genomes, 5mC and 5hmC. FIG. 1B) Top: The epigenetic code reveals cell identity. Bottom: Strengths and challenges for sequencing DNA including cell-free DNA (cfDNA) with various methods.

FIG. 2A-2C: Resistant cytosines can be built into DNA molecules that can be ligated to DNA samples in the form of adapters. FIG. 2A. Natural cytosine variants are not compatible with enzymatic deamination, while bulky modifications to the 5-position make the cytosine resistant to enzymatic deamination. Included are N4- and C5-position modified cytosines as examples of natural and unnatural cytosines that meet the criteria of being bulky and obstructing enzymatic deamination. FIG. 2B. These resistant cytosines can be built into DNA molecules that can be ligated to DNA samples in the form of adapters. The sequences of a few representative adapters compatible with next-generation sequencing are shown at bottom, where the X modification involved the modified cytosine base and [i5], [i7] or [barcode] represent different indices or barcodes. SEQ ID NOS: 21, 22, full length adapters and SEQ ID NOS: 23, 24 stubby adapter variants, SEQ ID NO: 25 USER compatible stubby adapter and SEQ ID NO: 26, hairpin linker are shown. FIG. 2C. These resistant adapters can be modified with a binding partner, such as biotin, that enables epigenetic sequencing workflows on solid phase. Shown are examples of biotin being added either during synthesis, using analogs of biotin itself or nucleobase phosphoramidite precursors with biotin, enabling insertion of a modification into any site in the body or ends of the sequencing adapter. Alternatively, the adapter can be biotinylated post-synthetically using a polymerase and biotinylated nucleotide triphosphate, such as Biotin-16-Aminoallyl-2′-dUTP.

FIGS. 3A-3B: Sequencing adapter strategies and DNA deaminase-resistant adapters. FIG. 3A) Post-deamination adapter ligation library preparation. Adapter sequences can be ligated post deamination to avoid deamination of the adapters, but this process is time and resource consumptive. It also does not as easily allow for repetitive interrogation of the same DNA molecule as proposed in this document. FIG. 3B) Pre-deamination adapter ligation library preparation. Adapters that resist either chemical and/or enzymatic transformation can be adapted early in the library preparation and provide a streamlined workflow. FIG. 3C) Lambda genomic DNA was sheared and ligated with either unmodified adapters or stubby adapters fully modified with the specified cytosine analogs. The adapted DNA was then subjected to either no treatment or enzymatic deamination by A3A and library generation was attempted using primers that recognize the unmodified adapters. Top: An experimental schematic is provided. Bottom: qPCR data is provided from amplification with primers that bind adapter candidates following either no treatment or enzymatic deamination. The results show that C and 5mC adapters, commonly used, do not permit enzymatic deamination, while modified adapters resistant to enzymatic DNA deamination permit library generation.

FIG. 4A-4C: Enzymatic deamination can occur on solid-phase immobilized DNA. FIG. 4A) Experimental design for assessing deamination of immobilized DNA. DNA was adapted akin to FIG. 3C, but now with biotinylated adapters. The DNA was immobilized on a bead and then denatured with NaOH washes. Amplification was carried out with primers internal to the DNA sequence that will amplify independent of deamination. The PCR products were then sequenced or assessed for cleavage using a restriction enzyme that interrogates one specific site inside the PCR amplicon. FIG. 4B) EditR window visualizing multiple sites (in disfavored sequence contexts for A3A deamination) with +/−NaOH used for denaturation. The red box below the Sanger trace highlights cytosine bases (SEQ ID NO: 27 top and SEQ ID NO: 28, bottom are shown. FIG. 4C) Digestion assay to interrogate deamination status of a single TCGA Taq^{α}I restriction site. Condition 1 represents a positive deamination control (S.C.=snap cool) while condition 6 is a negative deamination control with no NaOH wash. Conditions 2-5 are experimental, solid-phase immobilized deamination conditions interrogating different wash steps. The results show that snap cooling or NaOH based deamination of immobilized DNA can generate a substrate for enzymatic deamination and that enzymatic deamination can be successfully carried out on DNA immobilized on the solid phase.

FIG. 5A-5D: Modified adapters support enzymatic deamination based sequencing pipelines, including simultaneous genetic and epigenetic sequencing. FIG. 5A) The direct methylation sequencing (DM-Seq) pipeline makes use of modified DNA deaminase-resistant adapters and strand copying with a DNA polymerase and 5mC. Sheared gDNA is end-prepped and adapted to A3A-resistant 5pyC adapters. A copy strand made with 5mCTPs is synthesized before glucosylation and carboxymethylation. A3A deaminates 5mCpGs to Ts which can be detected upon PCR amplification. The method requires the obligate use of DNA deaminase-resistant adapters to act as primers for the copy strand step and to tolerate subsequent deamination. FIG. 5B) DM-Seq using 5-pyC adapters accurately detects 5mCpGs at single-base resolution and is more DNA sparing than BS-Seq. At left, Difference in Ct between DM-Seq and BS-Seq determined by qPCR. p-value represents paired two-tailed t-test (n=3 MTase conditions). In Middle, shown is the genome browser view for coordinates 24,000-28,000 in the lambda phage genome for all CpGs. Lambda gDNA was modified with SAM and no MTase, M.SssI (CpG), or M.CviPI (GpC). Numbers on left represent total efficiency across the entire 48.5 kB genome. At right, correlation of M.CviPI generated heterogeneously modified CpGs at single-base resolution. Only CpCpGs are plotted to quantify performance of DM-Seq vs BS-Seq at heterogeneously modified CpGs. FIG. 5C). Copying with DNA deaminase-susceptible or DNA deaminase-resistant dCTPs allows for different sequencing pipelines. Top. In DM-Seq, the stubby adapter acts as a primer binding site for the generation of a 5mC copy strand, which is not maintained through library preparation. In contrast, the strand could be maintained if an A3A-resistant dCTP analog was used to generate the copy strand. Library generation would then result in reads that are epigenetic reads, with converted cytosines, and genetic reads with unconverted cytosines. The two reads can be matched by the shared 5′- and 3′-ends or using barcodes. Bottom. In an analogous manner, a hairpin could be ligated to molecules and used to generate a DNA deaminase-resistant copy strand while also linking the two strands. FIG. 5D) A representative workflow for reading out genetic and epigenetic information. A hairpin is used to link the target strand, which is susceptible to enzymatic conversion, with a deamination-resistant copy strand. Single A-tail overhands are added to the extended, and thus blunt-ended molecule which can be used to ligate adapters containing resistant bases. These adapted molecules are first protected at 5hmCs by βGT and then deaminated by A3A. The whole molecule is read out where both epigenetic and genetic sequence information can be parsed. The method is distinguished from existing methods in the use of DNA deaminase-resistant adapters and copying with DNA deaminase-resistant dCTPs, which permits the all-enzymatic approach to simultaneous reading of epigenetic and genetic information.

FIGS. 6A-6B: Solid-phase immobilized substrate epigenetic sequencing workflows are more streamlined relative to solution-phase approaches. FIG. 6A) Generalized scheme of standard epigenetic sequencing which traditionally requires the use of DNA-binding Magnetic Bead (DMB) based purification, which relies on the affinity of DNA for the bead, and is time and effort consumptive. The scheme depicted starts with DNA that has already been sheared, end-repaired, and ligated to A3A-resistant adapters. In comparison, SMB substrate immobilization, which relies on tight interaction between the modified adapter and the solid-phase bound binding partner, allows for rapid purification between library preparation steps. FIG. 6B) Comparison of time required for DMB and SMB-based purifications.

FIGS. 7A-7B. Streamlined epigenetic sequencing performed on immobilized substrates has equivalent accuracy to sequencing performed onsolution-based substrates. FIG. 7A) Workflows for solid-phase APOBEC Coupled Epigenetic Sequencing (spACE-Seq) and resin-based Enzymatic Methylation Sequencing (rEM-Seq). FIG. 7B) Comparison of deamination efficiencies on control DNAs with various combinations of enzymatic steps on solid phase and solution-based substrates, demonstrates that enzymatic conversion steps with DNA deaminases, TET enzymes and glucosyltransferases are feasible on immobilized DNA. Thus, modified DNA deaminase resistant adapters permit the sequencing workflows to be carried out on immobilized DNA with high accuracy and greater efficiency.

FIGS. 8A-8G: Bisulfite and enzymatic-resistant adapters provide new opportunities for epigenetic sequencing to resolve 5mC and 5hmC. FIG. 8A) Schematic for bACE-Seq method for determining 5hmC and 5mC via a subtraction-based workflow. Conventional bACE-Seq does not allow for resolution of 5mC and 5hmC on the same DNA molecule. However, modified workflow with novel adapters enables this determination. FIG. 8B) Adapter candidates are assessed for resistance to both BS and A3A. FIG. 8C) Left. Adapters that are resistant to both BS/A3A enable a pre-deamination adapter workflow. Right. Data from a sequencing analysis using this pre-deamination adapter strategy is provided with different adapter candidates, demonstrating the specific deamination of 5mC after the second DNA deamination step. FIG. 8D) Multiplexed BS/A3A sequencing workflow for parsing of C, 5mC, and 5hmC in cis. FIG. 8E) Ternary code analysis via 5′ and 3′ end decoding allows for the translation of a standard sequencing binary code into a ternary code. FIG. 8F) Data demonstrating that methylated human DNA (fully methylated Jurkat T-cell line genomic DNA) is detected as either 5mC or 5hmC following BS and is determined to be 5mC following A3A treatment. An advantage of the solid-phase immobilized enzymatic deamination method is that the same DNA molecule can potentially be interrogated more than once in library constructs. DNA that has been treated with bisulfite leads to the conversion of C to U. 5mC is resistant to deamination, while 5hmC is converted to the adduct CMS. If this bisulfite-converted DNA is then enzymatically deaminated using A3A, the 5mC will convert to T, but the 5hmC (protected as CMS) will not. A library could be generated from the immobilized DNA after bisulfite and then again after A3A. The comparison of either molecular barcodes or matching molecules with the same unique 5′ and 3′ ends (as noted in the figure) could then be used the decode when 5mC and 5hmC are present on the original starting DNA molecule. The generation of two libraries from the same starting DNA is a distinctive potential advantage of deamination protocols on immobilized DNA, where multiple processes can take place with retention of the starting DNA molecules. FIG. 8G) A representative workflow that combines strategies from FIG. 5G with strategies from FIG. 8D. The result is the generation of a library where the status of C, 5mC, 5hmC can be parsed while a linked read maintains the original genetic code.

### DETAILED DESCRIPTION OF THE INVENTION

Nature offers a suite of enzymes with biological roles in cytosine modification spanning from bacteriophages to mammals. These enzymatic activities include methylation by DNA methyltransferases, oxidation of 5mC by TET family enzymes, hypermodification of 5hmC by glucosyltransferases, and the generation of transition mutations from cytosine to uracil by DNA deaminases. The present invention leverages the natural reactivities of these DNA-modifying enzymes and converts them into powerful biotechnological tools. More specifically, the application of these DNA-modifying enzymes in sequencing relies on their natural activities while also exploiting their ability to discriminate between cytosine modification states. We show that using cytosine analogs that are resistant to DNA deaminases provides significant advantages for rapid and efficient epigenomic sequencing, can be used to resolve multiple different DNA modification states in the same DNA molecule, or to simultaneously resolve genetic and epigenetic information.

Improved DNA methylation assays have a variety of applications, particularly in personalized medicine and forensic science [1]. The identification of epigenetic-based biomarkers for cancer and other epigenetic-related diseases, can provided the clinician with guidance as to the presence or severity of a disease, and streamline treatment options for the patient. As discussed below, DNA methylation assays can also be applied to the discrimination of fetal and maternal DNA in circulating cell-free DNA for downstream epigenetic sequencing analysis.

DNA methylation analysis can also be used for verification of DNA samples, body fluid identification and the estimation of ages and phenotypic characteristics.

“Liquid biopsies” can extract clinically actionable information from easily accessible bodily fluids, offering a potential replacement for informative but difficult to obtain surgical biopsies. As discussed above, oncoproteins, circulating tumor cells, and free-floating nucleic acids have been identified in plasma and provide promising sources for new biomarkers. Circulating “cell-free” DNA (cfDNA) is particularly compelling, as it contains nucleotide-specific information that can lead to changes in therapy. cfDNA quantity correlates with tumor stage and type, and FDA-approved cfDNA gene panels can track the emergence of resistance. As sensitive sequencing techniques improve, it is anticipated that somatic mutations will be detected at earlier stages of tumor evolution. However, mutational signatures can be shared between multiple tumors and are not always definitive for identifying the tissue-of-origin. Therefore, detection of ‘higher-order’ information beyond simple mutations will remain an unmet need in the absence of new, transformative technologies.

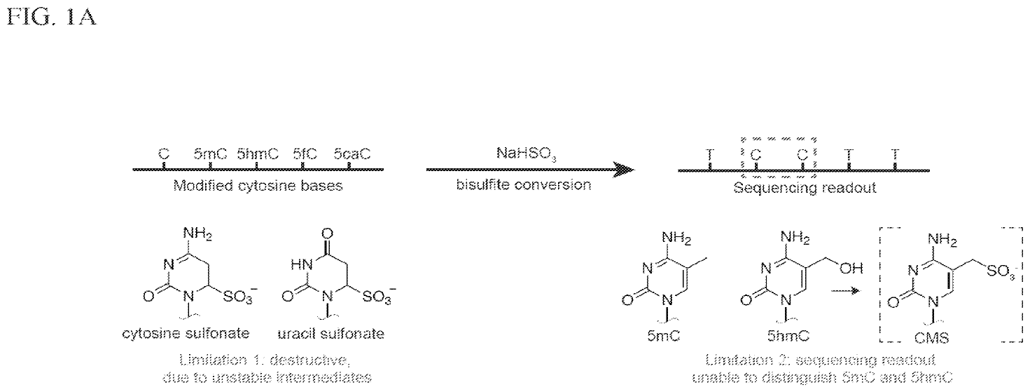

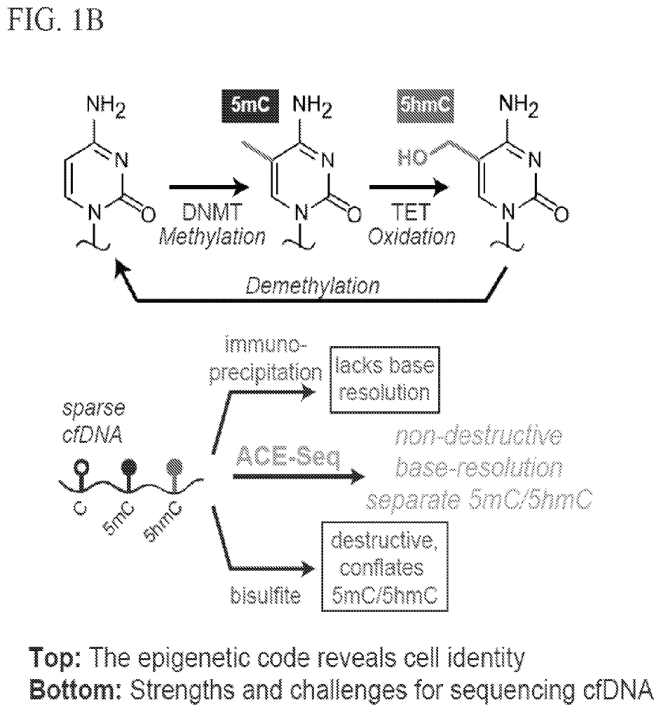

cfDNA contains such higher-order information in the form of epigenetic modifications, especially within Cytosine-Guanine (CpG) dinucleotides, which remain underexplored due to technological limitations (FIG. 1B). The most prevalent marker is cytosine methylation at the 5-position. Methylated CpGs (5mCpGs) are associated with silenced chromatin, and their signature, particularly in CpG rich islands (CGIs) and shores near promoters, can therefore define cell lineage. Although it was long believed that 5mC was the only such modification, the discovery of TET enzymes revealed the existence of other epigenetic CpG modifications. TET enzymes can oxidize 5mC to generate 5-hydroxymethylcytosine (5hmC), which can accumulate to levels as high as 40% of 5mC in certain cell types. Further oxidization of 5hmC also occurs, yielding bases that are exceptionally rare, but which can play a role in erasure of 5mC. The current model governing CpG modifications implicates methylation and oxidation together in a cycle of modification and de-modification that can regulate gene expression and define cellular identity [14].

### Definitions

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Suitable polynucleotides include DNA, preferably genomic DNA. The polynucleotides comprising the sample nucleotide sequence may be obtained or isolated from a sample of cells, for example, mammalian cells, preferably human cells. Suitable samples include isolated cells and tissue samples, such as biopsies.

The term “biological sample” includes, without limitation, cell-containing bodily fluids, peripheral blood, tissue homogenates, aspirates, and any other source of rare cells or polynucleotides that are obtainable from a human subject.

Modified cytosine residues including 5hmC and 5mC have been detected in a range of cell types including embryonic stem cells (ESCs) and neural cells. Suitable cells also include somatic and germ-line cells which may be at any stage of development, including fully or partially differentiated cells or non-differentiated or pluripotent cells, including stem cells, such as adult or somatic stem cells, cancer stem cells, fetal stem cells or embryonic stem cells.

For example, polynucleotides comprising the sample nucleotide sequence may be obtained or isolated from neural cells, including neurons and glial cells, contractile muscle cells, smooth muscle cells, liver cells, hormone synthesizing cells, sebaceous cells, pancreatic islet cells, adrenal cortex cells, fibroblasts, keratinocytes, endothelial and urothelial cells, osteocytes, and chondrocytes.

Cells of interest include disease-associated cells, for example cancer cells, such as carcinoma, sarcoma, lymphoma, blastoma or germ line tumor cells. Other cell types include those with a genotype of a genetic disorder such as Huntington's disease, cystic fibrosis, sickle cell disease, phenylketonuria, Down syndrome, or Marfan syndrome.

Polynucleotides to be assessed also include those present in cell-free circulating DNA present in circulation in serum and blood. Such DNA molecules can be associated with certain pathologies or can derived from the fetus in a pregnant woman. The compositions and methods disclosed herein are particularly amenable to analysis of sparse DNA samples.

Methods of extracting and isolating genomic DNA and RNA from samples of cells are well-known in the art. For example, genomic DNA or RNA may be isolated using any convenient isolation technique, such as phenol/chloroform extraction and alcohol precipitation, cesium chloride density gradient centrifugation, solid-phase anion-exchange chromatography and silica gel-based techniques.

In some embodiments, whole genomic DNA and/or RNA isolated from cells may be used directly as a population of polynucleotides as described herein after isolation. In other embodiments, the isolated genomic DNA and/or RNA may be subjected to further preparation steps. The genomic DNA and/or RNA may be fragmented, for example by sonication, shearing or endonuclease digestion, to produce genomic DNA fragments. A fraction of the genomic DNA and/or RNA may be used as described herein. Suitable fractions of genomic DNA and/or RNA may be based on size or other criteria. In some embodiments, a fraction of genomic DNA and/or RNA fragments which is enriched for CpG islands (CGIs) may be used as described herein.

The term, “epigenetics,” refers to the complex interactions between the genome and the environment that are involved in development and differentiation in higher organisms. The term is used to refer to heritable alterations that are not due to changes in DNA sequence. Rather, epigenetic modifications, or “tags,” such as DNA methylation and histone modification, alter DNA accessibility and chromatin structure, thereby regulating patterns of gene expression. These processes are crucial to normal development and differentiation of distinct cell lineages in the adult organism. They can be modified by exogenous influences, and, as such, can contribute to or be the result of environmental alterations of phenotype or pathophenotype. Importantly, epigenetic programming has a crucial role in the regulation of pluripotency genes, which become inactivated during differentiation.

The term “methylation” of DNA, refers to DNA modifications, typically found on cytosine bases. The term “modified” DNA and “methylated” DNA can be used interchangeably to refer to DNA that is methylated or hydroxymethylated, containing the bases 5-methylcytosine (5mC) or 5-hydroxymethylcytosine (5hmC) in various combinations, or to contain additional natural modifications of 5mC.

The terms “construct”, “cassette”, “expression cassette”, “plasmid”, “vector”, or “expression vector” is understood to mean a recombinant polynucleotide, generally recombinant DNA, which has been generated for the purpose of the expression or propagation of a nucleotide sequence(s) of interest or is to be used in the construction of other recombinant nucleotide sequences.

“DNA Deaminases” are enzymes that deaminate unmodified or subsets of modified cytosines. Notable chemical means for deamination are known and stand in contrast. Unmodified cytosine can be deaminated by the chemical bisulfite, as can 5fC and 5caC. Borane-mediated conversion to dihydrouracil represents another mechanism for deaminating 5caC. However, an enzymatic alternative exists for achieving similar results. The DNA deaminases of the AID/APOBEC family play critical functions in adaptive or innate immunity, initiating antibody maturation and restricting retroviruses from replicating. In their canonical roles, AID/APOBECs use a zinc cofactor to activate water for nucleophilic attack on cytosines in single-stranded DNA (ssDNA). Enzymatic deamination by activated nucleophilic attack thus bypasses the unstable sulfonated intermediate generated in bisulfite-based deamination.

A series of findings suggesting that DNA deaminases can discriminate between different cytosine modification states revealed new possibilities for their application in sequencing pipelines. The initial detection of activity on 5mC led to conjecture about possible moonlighting roles for DNA deaminases in epigenetic reprogramming. Subsequent systematic studies revealed that while activity on unmodified C and 5mC can be readily detected, deamination activity against 5hmC is significantly impaired [15]. Based on the analysis of a larger series on natural and unnatural 5-position modified cytosines, the mechanistic basis for discrimination appeared to be selection against bulky or electronegative substituents. This trend was maintained with APOBEC3A (A3A), the most active of AID/APOBEC deaminases, and extended to discrimination against 5fC and 5caC [16]. Crystal structures have provided a molecular rationale for discrimination against larger 5-position substrates, with an active site residue (Tyr130) positioned to act as a hydrophobic gate adjacent to the C5-C6 face of cytosine in the structure of A3A bound to ssDNA [16,17].

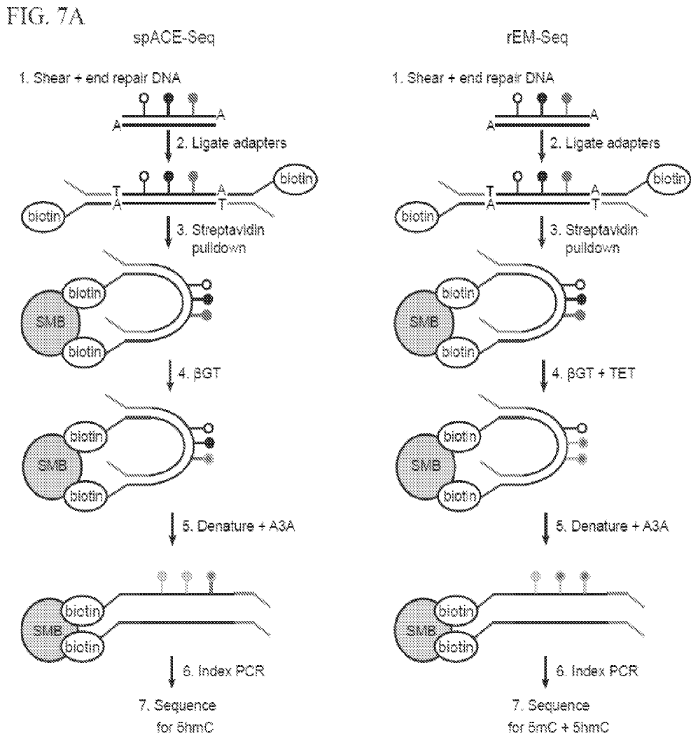

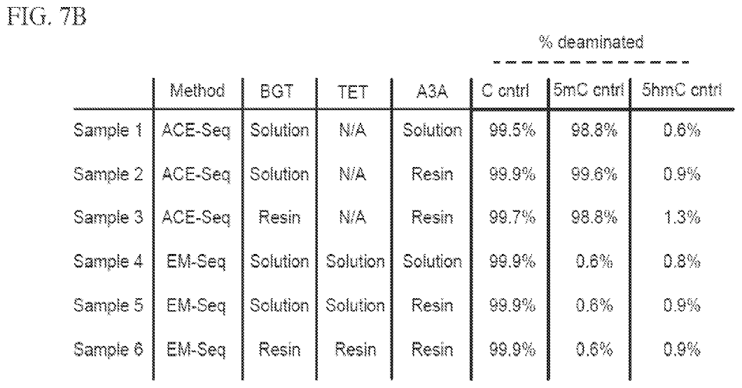

Grounded in these extensive biochemical and structural studies, A3A has now been used in various approaches for epigenetic sequencing, all linked by their common reliance on discrimination against bulky 5-position-modified cytosine bases. Sequencing using enzymatic DNA deamination was pioneered in APOBEC-Coupled Epigenetic Sequencing (ACE-Seq) (FIG. 7A) [18]. In this strategy, all 5hmCs are first converted to 5ghmC by T4-βGT. Adding bulk to 5hmC blocks low level deamination, and the remaining unmodified C and 5mC can be efficiently deaminated by A3A. ACE-seq represents the first non-destructive sequencing approach for profiling 5hmC at base resolution and additionally shows a sensitivity and specificity that outpaces bisulfite-based approaches.

A3A has also been combined with both TET enzymes and T4-βGT in a method first proposed [18] and then further independently developed by Vaisvila et al. called Enzymatic Methylation Sequencing (EM-Seq) [19]. In this approach, genomic DNA is oxidized by TET enzymes in the presence of T4-βGT. The 5mC and 5hmC are thus converted to a combination of 5caC and 5ghmC. As these modified bases are resistant to A3A-mediated deamination, subsequent treatment with A3A results in deamination of only unmodified cytosines, providing a readout akin to standard bisulfite. Importantly, this method has been extended to long read platforms, such as PacBio and Nanopore, taking advantage of the non-destructive nature of enzymatic deamination [20].

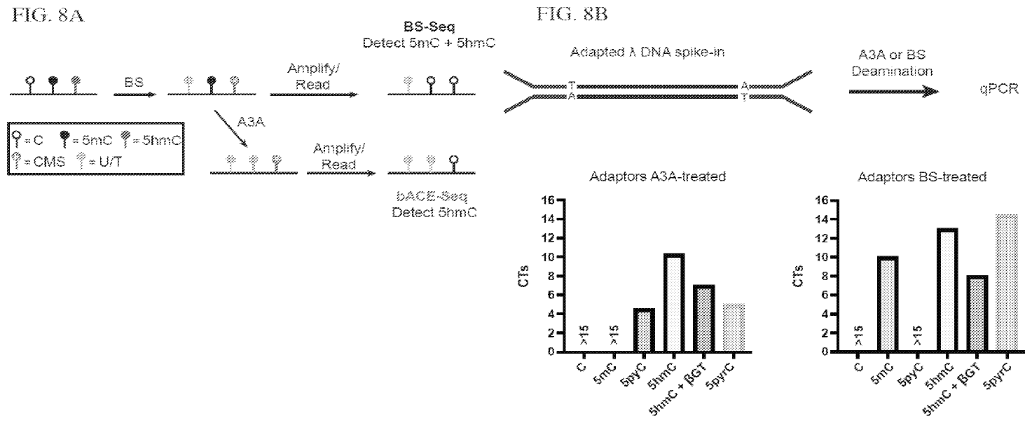

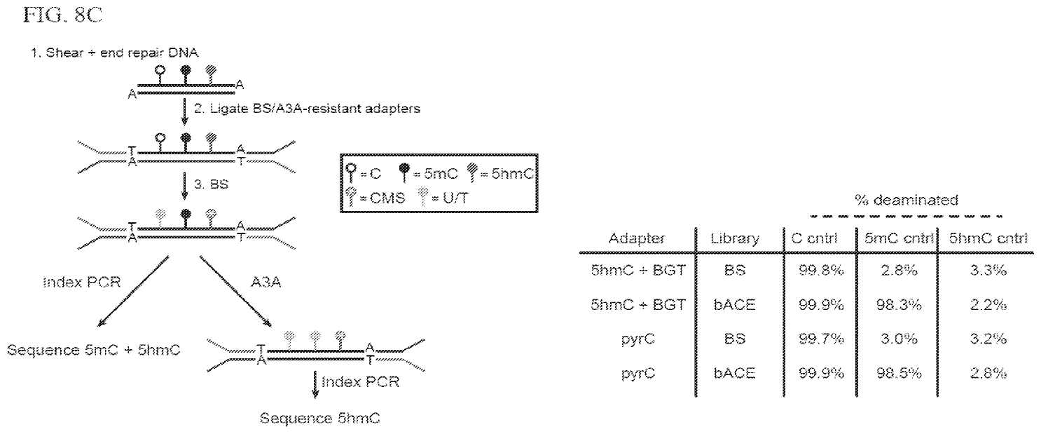

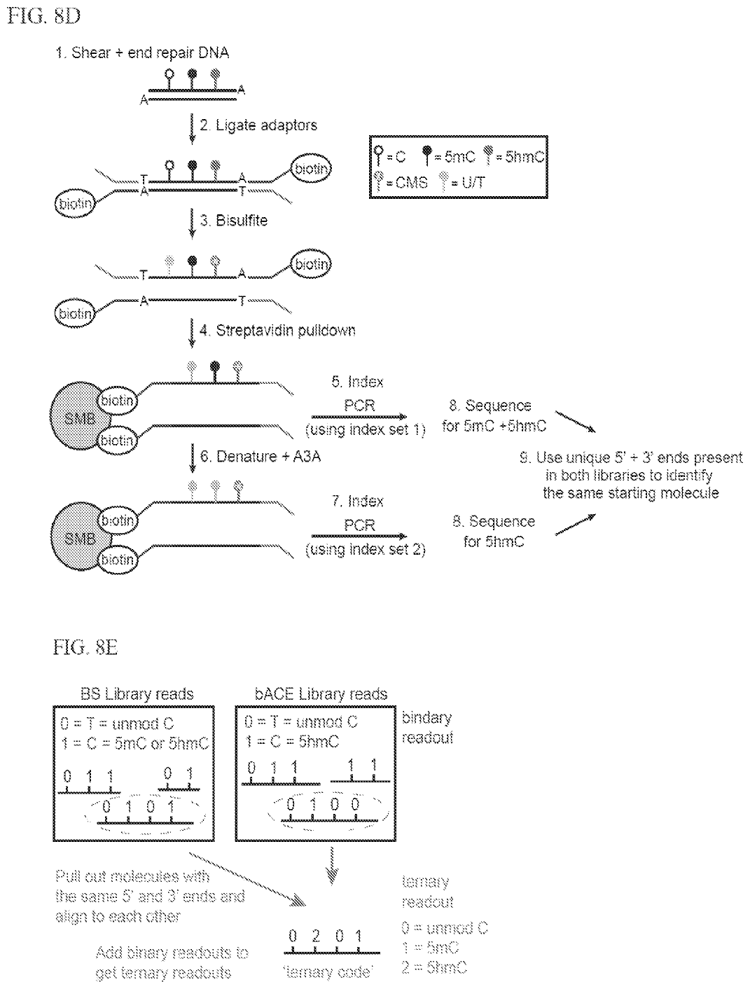

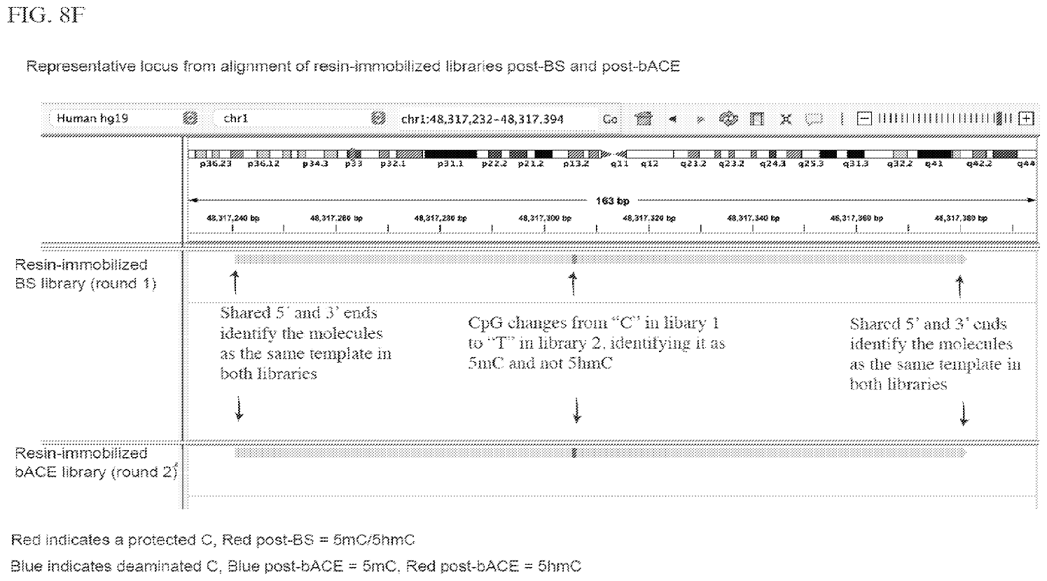

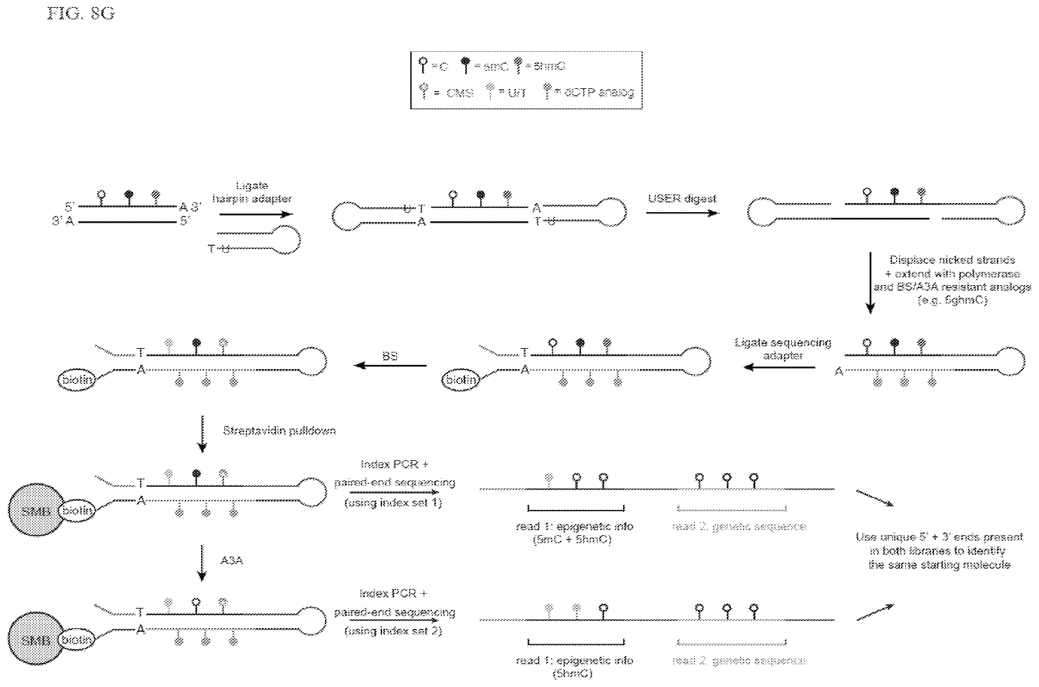

Enzymatic deamination has also been combined with bisulfite in a manner that exploits the differential reactivity of 5mC and 5hmC [21]. Bisulfite and APOBEC-Coupled Epigenetic Sequencing (bACE-Seq), builds on the fact that although 5hmC does not deaminate, the reaction to form CMS creates a bulky 5-position adduct that makes the modified base resistant to enzymatic deamination (FIG. 1, FIG. 8A). Added benefit comes from the fact that bisulfite can simultaneously fragment DNA and yield the ssDNA substrate needed for enzymatic deamination. In bACE-Seq, after treatment with bisulfite, the DNA can be split into two parallel workflows: one to detect 5mC and 5hmC together (BS-only), and the other treated with A3A to deaminate 5mC, leaving only original 5hmC bases reading as C. Thus, the ability for DNA deaminases to discriminate between cytosine modifications has already been exploited to great effect, with a promise of more innovations to come. Nonetheless, it was previously unknown whether DNA deaminase enzymes can work on immobilized DNA substrates.

“Deamination” is the removal of an amino group from a molecule. Enzymes that catalyze this reaction are called deaminases. Deaminases include, without limitation, APOBEC1, APOBEC3A, APOBEC3B, APOBEC3C, APOBEC3DE, APOBEC3F, APOBEC3G, Activation-induced cytidine deaminase (AID), and CDA from lamprey. More broadly this deaminase family includes homologs from various species all of which are thought to catalyze similar reactions on nucleic acids as described [22,23].

“Glucosyltranferases” are a group of enzymes that catalyze the transfer of glucosyl groups in biochemical reactions. Phage-derived T4 β-glucosyltransferase (referred to as βGT or BGT throughout) has been employed in enrichment-based or near base-resolution detection of 5hmC in genomic samples. hmC-Seal was the first enzymatic enrichment-based approach for studying 5hmC [24]. In this approach, the native T4-βGT is used, but with an unnatural substrate—a chemically-modified UDP-glucose derivative containing an azide functional group (UDP-6-azide-glucose)—that site-specifically labels all 5hmC bases with the azido-modified glucose.

Two types of approaches leveraging the phage-derived T4 β-glucosyltransferase (βGT) have been developed, which permit either enrichment-based or near base-resolution detection of 5hmC in genomic samples hmC-Seal was the first enzymatic enrichment-based approach for studying 5hmC [24]. In this approach, the native T4-βGT is used, but with an unnatural substrate—a chemically-modified UDP-glucose derivative containing an azide functional group (UDP-6-azide-glucose)—that site-specifically labels all 5hmC bases with the azido-modified glucose. The azido group can then be conjugated to a biotin-containing alkyne using copper-free click chemistry. The canonical biotin-streptavidin interaction is then exploited to enrich for molecules containing 5hmC bases in a manner analogous to an antibody pulldown experiment. These molecules can then be PCR amplified. Subsequent optimizations of this method have been able to obtain information from as few as 1000 cells and have been explored as cancer diagnostic when applied to cell-free circulating DNA [25-28].

A recent derivative technique named Jump-Seq also starts with utilizing T4-βGT to label 5hmC with an azido-modified glucose [29]. However, rather than biotin, the subsequent click chemistry tags the 5hmC-containing DNA with a hairpin oligonucleotide. This hairpin can then prime polymerase extension and, due to the covalent tether, the extended DNA can “jump” onto a 5hmC landing site. The technique can be used to infer near base resolution information of 5hmC in a cost-effective manner. A similar approach called hmTOP-Seq makes use of a tethered oligonucleotide as the template for primed extension and 5hmC localization [30].

“Ten-eleven translocation methylcytosine dioxygenases (TET)” comprise a family of enzymes involved in DNA demethylation and therefore gene regulation [8,31]. TET2, for example, catalyzes the conversion of the modified DNA base 5mC to 5hmC. TET2 produces 5hmC by oxidation of 5mC in an iron and alpha-ketoglutarate dependent manner. The conversion of 5mC to 5hmC has been proposed as the initial step of active DNA demethylation in mammals. Additionally, downgrading TET2 has decreased levels of 5-formylcytosine (5fC) and 5-carboxylcytosine (5caC) in both cell cultures and mice. Notably, a site with a 5hmC base already has increased transcriptional activity, a state termed “functional demethylation”. This state is common in post-mitotic neurons.

The discovery that bisulfite is unable to distinguish between 5mC and 5hmC [12] motivated efforts to separate the detection of these two bases with chemical or enzymatic approaches. These efforts have relied upon the fact that 5fC and 5caC are both generally susceptible to bisulfite-mediated deamination, although it is important to note that the efficiency of 5fC deamination is not as high as unmodified cytosine.

An early orthogonal approach used a combination of enzymatic approaches with bisulfite. In their native role, TET enzymes catalyze the Fe(II)- and α-ketoglutarate-dependent oxidation of 5mC to 5hmC, 5hmC to 5fC, and 5fC to 5caC. In Tet-Assisted Bisulfite Sequencing (TAB-Seq) [32,33], the activities of TET on 5mC and 5hmC are uncoupled from one another by first quantitatively converting all 5hmC to 5ghmC with UDP-glucose and T4-βGT. These 5ghmC bases are then subsequently protected from TET-mediated oxidation, while 5mC bases are oxidized to 5fC or 5caC. Subsequent bisulfite treatment renders only the original 5hmC bases resistant to deamination. While a single TAB-Seq experiment allows for the user to sequence 5hmC as C, comparison with standard bisulfite sequencing experiment (5mC+5hmC) can allow the user to indirectly infer 5mC by bioinformatic subtraction. While this approach is useful for convenience, indirect subtraction-based methods increase error, akin to 5hmC detection with oxBS-Seq [34], and cannot be applied in single cells given the need to process through two independent sequencing pipelines. An added limitation of TET-dependent sequencing approaches is the efficiency of TET enzymes themselves. TET enzymes are required to efficiently convert 5mC to 5caC in these sequencing pipelines, however the enzymes also prone to self-inactivation given that their highly reactive Fe(IV)-oxo intermediates and the efficiency of oxidation wanes going from 5mC to 5hmC to 5fC.

TET enzymes have also recently been applied in concert with non-bisulfite-mediated chemical deamination schemes for localizing modifications [35,36]. TET-assisted pyridine borane sequencing (TAPS) starts with TET-catalyzed oxidation of 5mC to 5fC or 5caC. When the genomic DNA is subsequently treated with pyridine borane, 5fC and 5caC are converted to dihydrouracil, a non-aromatic uracil analog which sequences as a T. The net result is a direct strategy for sequencing 5mC and 5hmC as T, while leaving unmodified C intact. A similar borane reduction strategy has also been combined with either T4-βGT (TAPSβ) or with potassium ruthenate (CAPS) to sequence 5mC and 5hmC individually, with varying degrees of efficiency. Notably, borane-mediated deamination requires lengthy incubation under acidic conditions but functions by a different mechanism that may be less destructive than bisulfite deamination, which is inherently dependent on unstable sulfonated intermediates.

“DNA methyltransferases” are a large group of enzymes that all methylate their substrates but can be split into several subclasses based on their structural features. The most common class of methyltransferases is class I, all of which contain a Rossmann fold for binding S-Adenosyl-L-methionine. While cytosine modification occurs predominantly in the CpG context in mammals, there are cytosine MTases across phylogeny which can act in a variety of different sequence contexts, and enzymatic sequencing approaches have exploited bacterial, viral, and mammalian MTases [37].

The discovery of bacterial MTases with a preference for the canonical mammalian CpG site provided an initial tool for use in sequencing. M.SssI, derived from a Spiroplasma strain MQ1, is one such CpG-specific MTase [38]. In a strategy termed Methylase-Assisted Bisulfite Sequencing wild-type M.SssI is used to convert unmodified CpGs in genomic DNA samples into 5mCpGs [39]. Given that these newly-modified CpGs are now protected from deamination, as are the original 5mC and 5hmC, treatment with bisulfite then allows for the base resolution sequencing of 5fC and 5caC as the two remaining bases susceptible to bisulfite-mediated deamination.

MTases can also be intentionally engineered to accept SAM analogs as substrates. As first achieved with the M.HhaI MTase, alteration of the SAM recognition motif via mutagenesis at two conserved polar residues, often a glutamine and asparagine, to alanine allows for transfer of larger extended alkyl chains from modified SAM analogs. Mechanistically, while steric accommodation on the enzyme side is one requirement for analog transfer, a second requirement is a conjugated pi system in the SAM analog that facilitates transfer by increasing the electrophilicity of the transferable moiety [40].

This steric engineering strategy has been extended from M.HhaI to M.SssI to create the enzyme eM.SssI [41]. In this approach, eM.SssI is used to react unmodified CpGs with a SAM analog containing one of two hex-2-ynyl side chains termed either Ado-6-amine or Ado-6-azide. These derivatized cytosine bases can then be subsequently coupled by amine-NHS or azide-DBCO conjugation chemistries to tag the modified DNA with biotin. Subsequent streptavidin pulldowns then enrich for fragments of DNA that are part of the “unmethylome”.

eM.SssI has also been applied for other non-canonical MTase reactions. In the absence of SAM, some MTases have been used to directly derivatize 5hmC with alkylthio moieties that can be further enriched. It has also been previously shown that MTases can promote removal of certain 5-position modifications in vitro and in the absence of SAM. In a recently developed method, caCLEAR [42], WT M.SssI is first employed to methylate all unmodified CpGs, and 5hmC bases are protected by T4-βGT. Then, subsequent decarboxylation with eM.SssI in the absence of SAM “clears” 5caC residues, converting them to unmodified CpG. Finally, eM.SssI is used to install Ado-6-Azide on all the original 5caC residues, while original unmodified cytosines, 5mC, and 5hmC residues remain unreacted. The azide-labelled 5caC residues can then be clicked to an oligonucleotide hairpin whereby subsequent polymerase extension can yield fragments enriched for 5caC. Collectively, these results have shown that both WT and rational engineering of the Spiroplasma M.SssI have been useful for studying mammalian cytosine modifications.

In an added extension of MTase reactivity, our group has recently discovered MTases that can be engineered to take on neomorphic carboxymethyltransferase activity (CxMTases) [13]. Building on insights gleaned from the structure of the recently crystallized CpG MTase M.MpeI, we found that a single active site point mutation could allow for the sparse natural metabolite carboxy-SAM (CxSAM) to be efficiently accepted as a substrate in lieu of SAM. We can couple this unique activity to create an A3A resistant 5-carboxymethylcytosine (5cxmC) base at unmodified CpGs work well with our existing ACE-Seq workflow and create the first fully enzymatic sequencing workflow to directly sequence 5mC at base resolution.

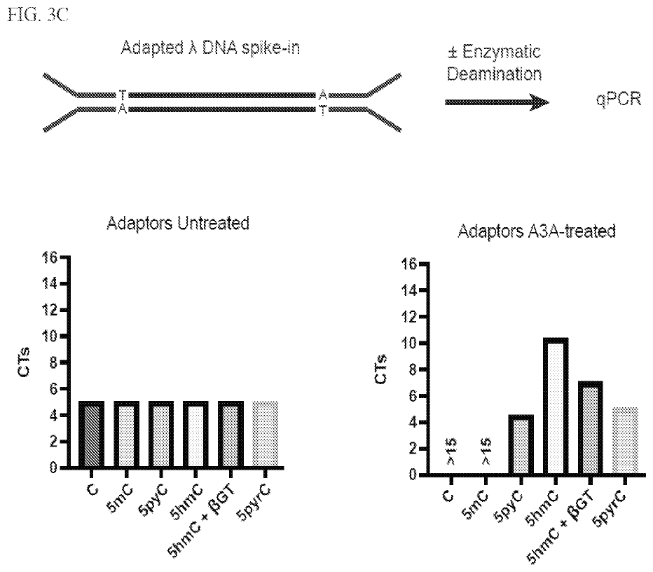

“DNA polymerases” are a large group of enzymes that are responsible for the DNA templated synthesis of DNA using deoxynucleotide triphosphates. DNA polymerases have numerous uses in sequencing pipelines, as the enzymes responsible for generation of DNA libraries and also as the enzymes that can be used to read the A, C, T and G bases on the DNA strand being sequenced. In the context of this document, DNA polymerases are discussed for their ability to copy DNA strands using not only the most common natural deoxynucleotide triphosphates (dNTPs), dATP, dCTP, dGTP and dTTP, but also modified dNTPs. Specifically, the use of modified dCTP analogs is described where the base modifications either render the cytosine susceptible to DNA deaminases (e.g., unmodified C or 5mC) versus those that render the cytosine resistant to DNA deaminases (e.g., 5pyC, 5pyrC, etc. as shown in FIG. 3C).

“DNA helicases” are a large group of enzymes that can unwind double stranded DNA to expose single stranded DNA. Helicases use the energy of ATP to move directionally along the duplex DNA and separate the two strands. In this document, helicases are also referred to as denaturing enzymes, given that they share function with other methods for denaturing duplex DNA, such as heat or chemical denaturants.

In general, “detecting”, “determining”, and “comparing” refer to standard techniques in epigenetic modification identification described in the examples and equivalent methods well known in the art. These terms apply particularly to sequencing, where DNA sequences are compared. There are a number of sequencing platforms that are commercially available and any of these may be used to determine or compare the sequences of polynucleotides.

The term “sodium bisulfite sequencing reagents” refers to prior art methods for detecting 5mC as is described in Frommer, et al., Proceedings of the National Academy of Sciences, 89.5:1827-1831 (1992) [7].

Solid-phase reversible immobilization, or SPRI, refers to a method of purifying nucleic acids from solution. It uses silica- or carboxyl-coated paramagnetic beads, which reversibly bind to nucleic acids in the presence of polyethylene glycol and a salt. A common application of SPRI technology is purifying samples of DNA amplified by PCR for sequencing reactions. SPRI as used in this document refers to direct DNA binding to magnetic beads (DMB) via charge interactions as opposed to the methods disclosed herein which rely upon interactions between specific binding pairs as described herein.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “comparison window” refers to a segment of at least about 20 contiguous positions in which a sequence may be compared to a reference sequence of the same number of contiguous positions after the two sequences are aligned optimally. In a refinement, the comparison window is from 15 to 30 contiguous positions in which a sequence may be compared to a reference sequence of the same number of contiguous positions after the two sequences are aligned optimally. In another refinement, the comparison window is usually from about 50 to about 200 contiguous positions in which a sequence may be compared to a reference sequence of the same number of contiguous positions after the two sequences are aligned optimally.

The terms “complementarity” or “complement” refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

The phrase “solid support” or “solid matrix” refers to any format, such as beads, microparticles, a microarray, the surface of a microtitration well or a test tube, a dipstick, a microwell plate, container, or a filter, and can also be referred to as “resin”. A solid matrix can comprise nucleic acids immobilized thereon such that they are not removable from the matrix in solution.

A bead may be porous, non-porous, solid, semi-solid, semi-fluidic, fluidic, and/or any combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μM), 5 μM, 10 μM, 20 μM, 30 μM, 40 μM, 50 μM, 60 μM, 70 μM, 80 μM, 90 μM, 100 μM, 250 μM, 500 μM, 1 mm, or greater. In some cases, a bead may have a diameter of less than about 10 nm, 100 nm, 500 nm, 1 μM, 5 μM, 10 μM, 20 μM, 30 μM, 40 μM, 50 μM, 60 μM, 70 μM, 80 μM, 90 μM, 100 μM, 250 μM, 500 μM, 1 mm, or less. In some cases, a bead may have a diameter in the range of about 40-75 μM, 30-75 μM, 20-75 μM, 40-85 μM, 40-95 μM, 20-100 μM, 10-100 μM, 1-100 μM, 20-250 μM, or 20-500 μM.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

The solid matrix, (e.g., beads) may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some embodiments, the solid support can be a functionalized magnetic particle. In some embodiments, the magnetic particle is a paramagnetic particle. The preferred magnetic particles for use in carrying out this invention are particles that behave as colloids. Such particles are characterized by their sub-micron particle size, which is generally less than about 200 nanometers (nm) (0.20 microns), and their stability to gravitational separation from solution for extended periods of time. In addition to the many other advantages, this size range makes them essentially invisible to analytical techniques commonly applied to cell and nucleic acid analysis. Particles within the range of 90-150 nm and having between 70-90% magnetic mass are contemplated for use in the present invention.

Suitable magnetic particles are composed of a crystalline core of superparamagnetic material surrounded by molecules which are bonded, e.g., physically absorbed or covalently attached, to the magnetic core and which confer stabilizing colloidal properties. The coating material should preferably be applied in an amount effective to prevent non-specific interactions between biological macromolecules found in the sample and the magnetic cores. Such biological macromolecules may include sialic acid residues on the surface of non-target cells, lectins, glycoproteins, and other membrane components. In addition, the material should contain as much magnetic mass/nanoparticle as possible. The size of the magnetic crystals comprising the core is sufficiently small that they do not contain a complete magnetic domain. The size of the nanoparticles is sufficiently small such that their Brownian energy exceeds their magnetic moment. Consequently, North Pole, South Pole alignment and subsequent mutual attraction/repulsion of these colloidal magnetic particles does not appear to occur even in moderately strong magnetic fields, contributing to their solution stability. Finally, the magnetic particles should be separable in high magnetic gradient external field separators. That characteristic facilitates sample handling and provides economic advantages over the more complicated internal gradient columns loaded with ferromagnetic beads or steel wool. Magnetic particles having the above-described properties can be prepared by modification of base materials described in U.S. Pat. Nos. 4,795,698, 5,597,531 and 5,698,271.

In some embodiments, at least a subset of the at least two different types of components or derivatives thereof are attached to the bead or the particle. In some embodiments, the at least a subset of the at least two different types of components or derivatives thereof are attached to the bead or the particle via suitable linkers used in the art. In some embodiments, one or more reagents for processing the components are attached to the beach or the particle. In some embodiments, the one or more reagents comprise one or more nucleic acid molecules. In some embodiments, the nucleic acid molecule comprises an adapter with 5pyC, 5pyrC or 5hmC. In some embodiments, the one or more reagents are attached to beads.

The term “specific binding pair” as used herein includes streptavidin-biotin, avidin-biotin, biotin analog-avidin, desthiobiotin-streptavidin, desthiobiotin-avidin, iminobiotin-streptavidin, iminobiotin-avidin, antigen-antibody, receptor-hormone, receptor-ligand, agonist-antagonist, lectin-carbohydrate, nucleic acid (RNA or DNA) hybridizing sequences, Fc receptor or mouse IgG-protein A, and virus-receptor interactions. In this document, “SMB” refers to a streptavidin conjugated magnetic bead.

“Positive selection” refers to purification from a mixture of different attachment of a first member of a specific binding pair that selectively binds to the second member of a second binding pair present on the target cell type or nucleic acid of interest, thereby allowing the cell or nucleic acid to be isolated from the mixture. A variety of means and methods for performing positive selections, i.e., purifying the entity of interest, employing the second member of a specific binding pair are well known in the art.

“Negative selection” refers to purification of a target cell type or nucleic acid from a mixture of different cell types by attachment of one or more first members of one or more specific binding pairs to each and every cell type or nucleic acid in the mixture with the exception of the cell type or target nucleic acid of interest. Specific binding pair reactions employing the second member of a binding pair allow those entities bearing the first member of a binding pair to be separated from the mixture, leaving behind the entity of interest. Means and methods for performing such separations are well known in the art. The portion of the mixture that is left behind is referred to as the negative fraction.

“Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them, and they are used interchangeably herein. The term “adapter” may also be used interchangeably with the terms “adaptor”, “oligonucleotide”, and “polynucleotide.” The term “adapter” can refer to a sequence of DNA that permits a DNA molecule to be sequenced on a given sequencing platform. An adapter may also comprise a hairpin linker, such as that used in hairpin bisulfite to tether two strands of DNA together [43,44].

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification but may alternatively be double-stranded. If double-stranded, the primer can first be treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al, editors, PCR: A Practical Approach and PCR2: A Practical Approach (IRL Press, Oxford, 1991 and 1995, respectively).

“Nested PCR” refers to a two-stage PCR wherein the amplicon of a first PCR becomes the sample for a second PCR using a new set of primers, at least one of which binds to an interior location of the first amplicon. As used herein, “initial primers” or “first set of primers” in reference to a nested amplification reaction mean the primers used to generate a first amplicon, and “secondary primers” or “second set of primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g., Bernard et al. Anal. Biochem., 273: 221-228 (1999) (two-color real-time PCR). Usually, distinct sets of primers are employed for each sequence being amplified.

The term “barcode” refers to a nucleic acid sequence that is used to identify a single cell, a subpopulation of cells, or a target nucleic acid. Barcode sequences can be linked to a target nucleic acid of interest during amplification and used to trace back the amplicon to the cell from which the target nucleic acid originated. A barcode sequence can be added to a target nucleic acid of interest during amplification by carrying out PCR with a primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid. In some context, the term barcode is used to refer to DNA that is characterized by unique fragmentation endpoints, as unique 5′- and 3′-ends of a DNA molecule can be characteristic when a DNA molecule is generated from longer DNA fragments that are subjected to fragmentation by enzymatic or mechanical methods.

The term “molecular identifier” (or “MID”) as used herein refers to a unique nucleotide sequence that is used to distinguish between a single cell or genome or a subpopulation of cells or genomes, and to distinguish duplicate sequences arising from amplification from those which are biological duplicates. MIDs may also be used to count the occurrences of specific, tagged sequences for absolute molecular counting. A MID can be linked to a target nucleic acid of interest by ligation prior to amplification, or during amplification (e.g., reverse transcription or PCR), and used to trace back the amplicon to the genome or cell from which the target nucleic acid originated. A MID can be added to a target nucleic acid by including the sequence in the adapter to be ligated to the target. A MID can also be added to a target nucleic acid of interest during amplification by carrying out reverse transcription with a primer that contains a region comprising the barcode sequence and a region that is complementary to the target nucleic acid such that the barcode sequence is incorporated into the final amplified target nucleic acid product (i.e., amplicon). The MID may be any number of nucleotides of sufficient length to distinguish the MID from other MID. For example, a MID may be anywhere from 4 to 20 nucleotides long, such as 5 to 11, or 12 to 20. In particular aspects, the MID has a length of 8 random nucleotides.

The terms “molecular identifier,” “MID,” “molecular identification sequence,” “MIS,” “unique molecular identifier,” “UMI,” “molecular barcode,” “molecular identifier sequence”, “molecular tag sequence” and “barcode” are used interchangeably herein.

A “selected phenotype” refers to any phenotype, e.g., any observable characteristic or functional effect that can be measured in an assay such as changes in cell growth, proliferation, morphology, enzyme function, signal transduction, expression patterns, downstream expression patterns, reporter gene activation, hormone release, growth factor release, neurotransmitter release, ligand binding, apoptosis, and product formation. Such assays include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmitter release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs. A candidate gene is “associated with” a selected phenotype if modulation of gene expression of the candidate gene causes a change in the selected phenotype.

### Kits for Practicing the Methods of the Invention

In a further aspect, a kit comprising a modified oligonucleotide comprising an adapter operably linked to a first member of a specific binding pair, wherein said adapter renders the oligonucleotide resistant to deamination is provided. The kit can also contain a solid support operably linked to a second member of the specific binding pair, which when incubated together forms a DNA containing binding complex. In certain embodiments, the solid support provided may be a container or set of containers (e.g. multi-well PCR plate or PCR tubes) where the surface is coated in a second member of the specific binding pair which can be used to capture the adapter conjugated target DNA. In cases where the solid support is a magnetic particle, the kit can also include the appropriate magnetic separator. In certain embodiments, the kit can also comprise other reagents and enzymes useful in the methods described above to identify the epigenetic modifications described herein. In particular, these kits can be used in a method for identifying methylated cytosine molecules in target nucleic acids in a rapid and efficient manner.

The following materials and methods are provided to facilitate the practice of the present invention.

### Materials and Methods

The protein purification of either the isolate A3A domain or MBP-A3A-His have been described previously [45].

### Adapters:

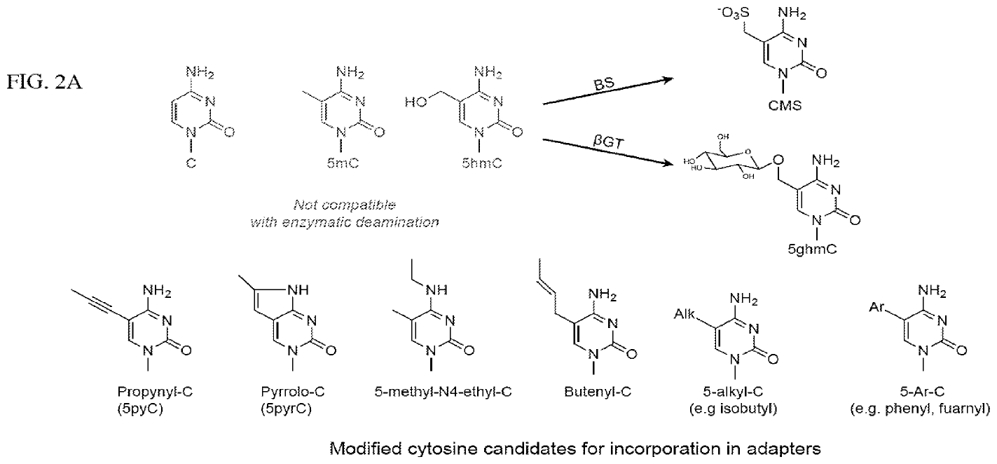

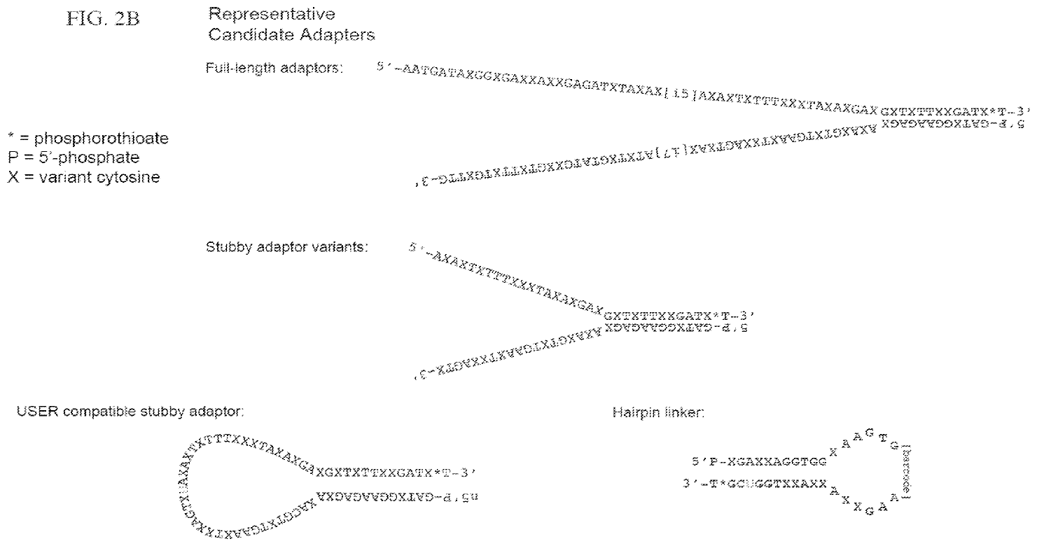

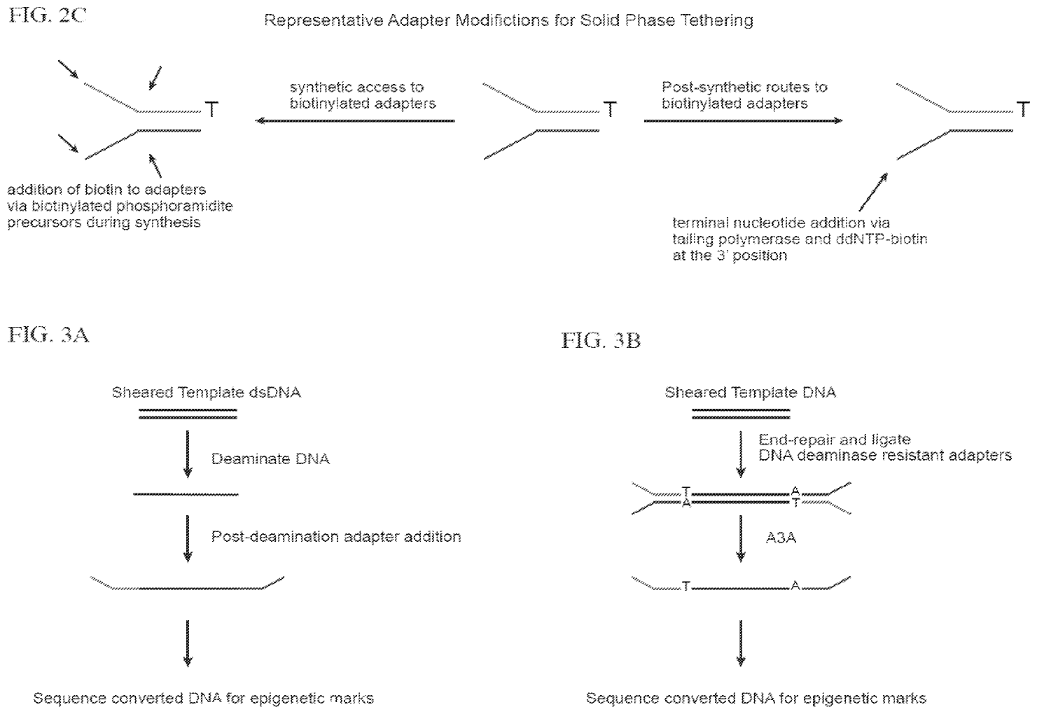

DNA oligonucleotides forming the adapters were synthesized by standard phosphonamidite chemistry by commercial vendors (Integrated DNA Technologies, IDT or Biomers). Some non-standard building blocks for synthesis were obtained from Glen Research. The two oligonucleotides that make up the adapter duplex were synthesized separately and annealed by standard protocols. The biotin tag on the adapter was introduced synthetically or enzymatically (see FIG. 2C). For enzymatic additions, DNA oligonucleotides were synthesized and then post-synthetically labeled with on the 3′ end using terminal transferase (TdT) from New England Biolabs (NEB) and Biotin-16-(5-aminoallyl)-ddUTP (Jena).

Some representative adapter sequences explored in this document include (see FIG. 2):

SA1-propynyl
(SEQ ID NO: 1)
5′-AXAXTXTTTXXXTAXAXGAXGXTXTTXXGATX*T-3′
where X = Propynyl-dC (Glen Research 10-1014),
and * = phosphorothioate bond. Partnered with
SA2-propynyl
(SEQ ID NO: 2)
5′-P-GATXGGAAGAGXAXAXGTXTGAAXTXXAGTX-3′
where X = Propynyl-dC (Glen Research 10-1014),
P = 5′-phosphate. The methylated adapters are
identical to the above sequences with X =
(SEQ ID NOS: 3 and 4)
5-methyl-dC.
SA1-pyrrolo
(SEQ ID NO: 5)
5′-AXAXTXTTTXXXTAXAXGAXGXTXTTXXGATX*T-3′
where X = Pyrrolo-dC, and * = phosphorothioate
bond. Partnered with
SA2-pyrrolo
(SEQ ID NO: 6)
5′-P-GATXGGAAGAGXAXAXGTXTGAAXTXXAGTX-3′
where X = Pyrrolo-dC and P = 5′-phosphate .
SA1-5hmC
(SEQ ID NO: 7)
5′-P-GATXGGAAGAGXAXAXGTXTGAAXTXXAGT-3′
where X = 5hmC and P = 5′-phosphate. Partnered
with
SA2-5hmC
(SEQ ID NO: 8)
5′-AXAXTXTTTXXXTAXAXGAXGXTXTTXXGATX*T-3′
where X = 5hmC and * = phosphorothioate bond.

These are compared to matched DNA sequences with unmodified cytosine (C) or 5mC.
Other relevant oligonucleotides include:

### Oligonucleotides

| DNA | Sequence | Purpose |
|---|---|---|
| 254mer | gtcactcagATGTATAGAATGATGAGTTAGGTA | Generate DNA |
| GeneBlock | GTGTTGATATGGGTTATGAATGAAGTAGTC | substrate with |
| | GATCTTTCATCATATTCTAGATCCCTCTGA | homogenously |
| | AAAAATCTTCCGAGTTTGCTAGGCAGTGAT | modified cytosines |
| | ACATAACTCTTTTCCAATAATTGGGGAAGT | (SEQ ID NO: 9) |
| | CATTCAAATCTATAATAGGTTTCAGATTTA | |
| | ATTCTGACTGTAGCTGCTGAAACGTTGCGG | |
| | AGTGTTAAGGTATATGAGTAGATGATTGAT | |
| | TGGGTATGTTGATAAGTGTAgtcactcag | |
| OTF12 | ATGTATAGAATGATGAGTTAGGTAGTGTTG | Generate DNA |
| | ATATGGGTTATGAATGAAGTA | substrate with |
| | | homogenously |
| | | modified cytosines |
| | | (SEQ ID NO: 10) |
| OTR12 | TACACTTATCAACATACCCAATCAATCATC | Generate DNA |
| | TACTCATATACCTTAACACT | substrate with |
| | | homogenously |
| | | modified cytosines |
| | | (SEQ ID NO: 11) |
| OTF2_TruSeq | ACACTCTTTCCCTACACGACGCTCTTCCGA | Primers for installing |
| | TCTTTGATATGGGTTATGAATGAAGTA | Illumina overhangs |
| | | (SEQ ID NO: 12) |
| OTR2_TruSeq | GACTGGAGTTCAGACGTGTGCTCTTCCGAT | Primers for installing |
| | CTAGTGTTAAGGTATATGAGTAGATGA | Illumina overhangs |
| | | (SEQ ID NO: 13) |
| 163mer spike in | ATATAGTGTGTAATATTAAGGGAGAATTG | Generate 163mer spike |
| GeneBlock | GCTGCTGCCGCTAAAGATAGTTTAGATATG | in |
| | GAATGACCCGGGACGATACGTATTCAAAG | (SEQ ID NO: 14) |
| | GTATCATGAAACGTTGGTCATAATAGATG | |
| | ATTGAGATTTAAGTATTTGTTGAGTTGATG | |
| | TTGTTTATTGGCGCGC | |
| Spike_In_F | ATATAGTGTGTAATATTAAGGGAGAATTG | Generate 163mer spike |
| | GCTGCTGCCGCTAAAGATAGTTTAGATATG | in with modified CpGs |
| | GAATGACC/i5HydMe-dC/GGGACGATA/iMe- | (SEQ ID NO: 15) |
| | dC/GTATT/iMe-dC/AAAG | |
| Spike_In_R | GCGCGCCAATAAACAACATCAACTCAACA | Generate 163mer spike |
| | AATA | in |
| | | (SEQ ID NO: 16) |
| Spike_In_post_F | GTGTGTAATATTAAGGGAGAATTG | Post deamination |
| | | primers |
| | | (SEQ ID NO: 17) |
| Spike_In_post_R | AATAAACAACATCAACTCAACAAATA | Post deamination |
| | | primers |
| | | (SEQ ID NO: 18) |
| Spike_In_post_ | ACACTCTTTCCCTACACGACGCTCTTCCGA | Primers for installing |
| F_TruSeq | TCTGTGTGTAATATTAAGGGAGAATTG | Illumina overhangs |
| | | (SEQ ID NO: 19) |
| Spike_In_post_ | GACTGGAGTTCAGACGTGTGCTCTTCCGAT | Primers for installing |
| R_TruSeq | CTAATAAACAACATCAACTCAACAAATA | Illumina overhangs |
| | | (SEQ ID NO: 20) |

### Ligation of DNA to Adapters:

Addition of adapters can be done to either PCR product or to sheared genomic DNA samples. The purified PCR products are generated with Taq polymerase to generate the single A overhands needed for ligation. For experiments with a fixed length PCR product, the PCR product is derived from a 272 base pair template DNA was obtained as a GeneBlock from IDT. The PCR product is the 254 bp sequence (see Table above) generated using primers OTF12 and OTR12 and Taq Polymerase (NEB) and purified over oligonucleotide spin columns (Qiagen). For genomic DNA samples, lambda phage genomic DNA was sheared and used as previous described (Schutsky et al, Nat Biotech, 2018). After shearing the DNA was then end repaired with NEBNext Ultra End Prep Kit. Lambda DNA samples were then ligated with adapters containing all unmodified C, 5mC, 5pyC, 5hmC, 5hmC+βGT, or 5pyrC modifications using NEBNext Ultra II Prep Kit and then purified by SPRI beads (Beckman, 1.2×) prior to sequencing.

### Assessment of Adapter Resistance to Chemical and Enzymatic Deamination:

Lambda genomic DNA was analyzed for library construction and deamination efficiency using either bisulfite sequencing or enzymatic deamination (see FIG. 3C). Sheared lambda genomic DNA was ligated to the specified adapters and then subjected to either standard bisulfite-mediated deamination following manufacturer instructions (Diagenode) or enzymatic deamination was performed using standard snap-cooling followed by deamination by APOBEC3A as previously described (Schutsky et al, Nat Biotech, 2018; Wang, Luo, Kohli, Method Mol Bio, 2020). The adapter sequences were used in a qPCR reaction to attempt library generation after deamination. For the libraries that could be constructed, the samples were sequenced on an Illumina MiSeq (150 bp paired end reads) and analyzed for deamination efficiency. Reads were quality and length trimmed with Trim Galore! Reads were aligned with Bismark and deduplicated with Picard and analyzed for cytosine deaminase efficiency (frequency of C read as T).

### Enzymatic Deamination of DNA Immobilized on Solid Phase:

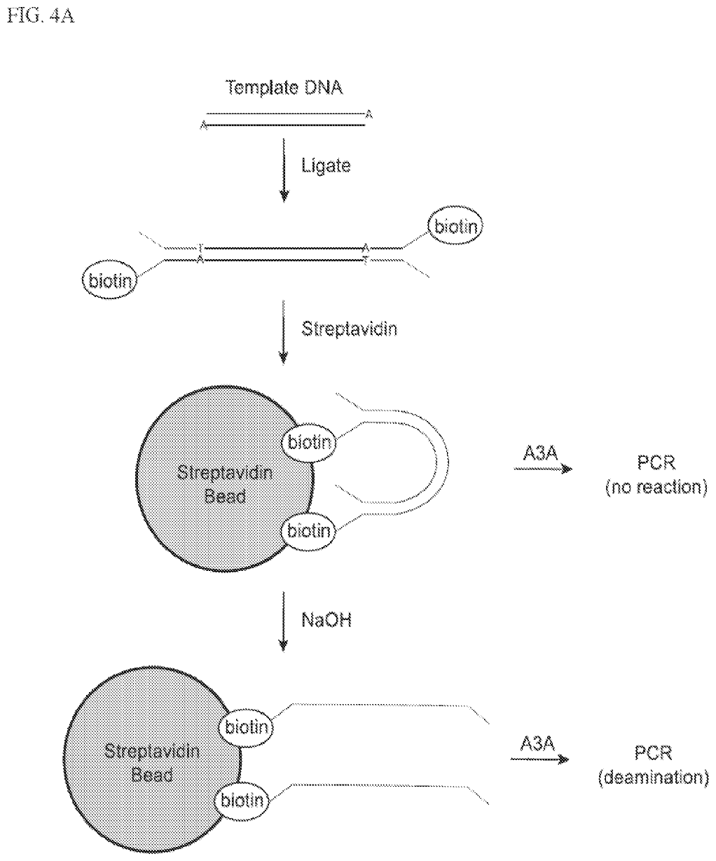

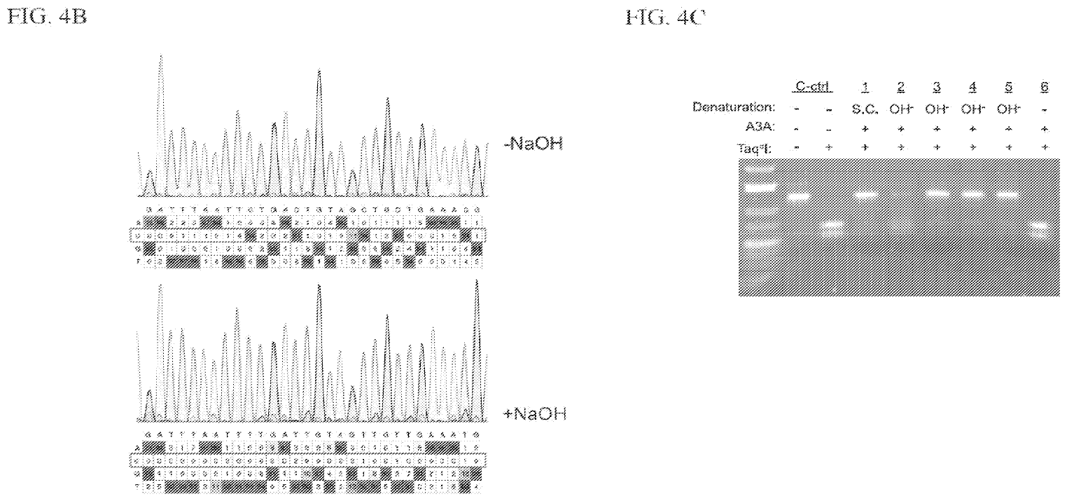

Modified DNA, either generated by PCR or sheared genomic sample, ligated with adapters containing a biotin, appended either synthetically or enzymatically as described above, was subjected to enzymatic deamination after immobilization on a solid phase (see FIG. 4A-C). The DNA was bound to streptavidin containing magnetic beads using standard protocols. After subjecting the DNA to either an NaOH (to denature the DNA) or wash buffer-only wash, the gDNA was then incubated at 37° C. for 1 hour with purified A3A using optimal buffer conditions. The bound DNA was then used as a template for PCR utilizing internal primers. The PCR products were Sanger sequenced and the traces were analyzed by EditR (http://baseeditr.com) (FIG. 4B) [46].

For analysis of gDNA (lambda phage), the 5pyC and biotin containing ligated lambda gDNA substrate was bound to solid phase and deaminated as above. As a control snap cooling of the resin was performed without incubation with A3A and samples were included with A3A without a NaOH wash. The bound DNA was used as a PCR template for amplification of a single locus within lambda gDNA that provides a readout of deamination efficiency. Within this amplicon, there is a single TCGA Taq^{α}I digestion site, which is resistant to cleavage if deamination occurs (generating a TTGA). Cleavage of the PCR product was attempted with Taq^{α}I under recommended conditions and the samples were run on an agarose gel for analysis (FIG. 4C).

### DM-Seq:

10 ng of gDNA ligated to 5pyC-containing adapters was used as input for DM-Seq. A methylated copy strand was created. 1 μM fully methylated primer was annealed in a total volume of 10 μL in CutSmart Buffer and 1 mM final concentration (individually) of dATP/dGTP/dTTP (Promega) and 5m-dCTP (NEB). 1 μl or 8 units Bst polymerase, large fragment (NEB) was added and incubated for 30 min at 65° C. The 5hmCs were then glucosylated with 40 μM UDP-Glucose and 1 μL or 10 units of T4 Phage β-glucosyltransferase (NEB) for 1 hour at 37° C. in a final volume of 20 μL. Incompletely copied or uncopied fragments were degraded with 1 μL or 10 units Mung Bean Nuclease (NEB) for 30 min at 30° C. After SPRI magnetic bead purification (1.2×), libraries were mixed with 0.5 μM MBP-M.MpeI-N374K and 160 μM CxSAM in carboxymethylation buffer (50 mM NaCl, 10 mM Tris-HCl pH 7.9, 10 mM EDTA) and incubated overnight at 37° C. followed by denaturation for 5 min at 95° C. 1 μL or 0.8 units of Proteinase K (NEB) was subsequently added and incubated at 37° C. for 15 min. The samples were purified using SPRI magnetic beads (1.2×) and eluted in 1 mM Tris-Cl, pH 8.0. DNA was then subjected to snap-cooling and A3A deamination in a final volume of 50 μL before SPRI magnetic beads purification (1.2×). DM-Seq libraries were amplified using indexing primers (IDT) and HiFi HotStart Uracil+ Ready Mix (KAPA Biosystems) before purification over SPRI magnetic beads (0.8×). Libraries were then characterized using a BioAnalyzer (High Sensitivity Kit, Agilent) and quantified (Qubit). For comparing performance relative to optimized DM-Seq, BS-Seq was performed on 10 ng gDNA ligated to 5mC-containing adapters (xGen, IDT), with no added copy or DM-Seq specific steps, using manufacturer instructions (Diagenode). Purified BS-Seq libraries were amplified using indexing primers (IDT) and HiFi HotStart Uracil+ Ready Mix (KAPA Biosystems) before purification over SPRI magnetic beads (0.8×) and ultimate characterization using a BioAnalyzer (High Sensitivity Kit, Agilent) and quantified (Qubit).

### Bioinformatics:

After sequencing of libraries either MiSeq or NextSeq instruments by standard protocols, reads were quality and length trimmed with Trim Galore! Reads were aligned with Bismark and deduplicated with Picard. Reads were filtered if 3 consecutive CpHs were non-converted using Bismark's existing filter_non_conversion command. Locus-specific amplicons (cytosine analog experiment, see above) were not deduplicated or filtered. Filtering served two purposes (in different experiments). For BS-Seq with copy-strand synthesis, the consecutive CpH conversion eliminated reads from copy-strand amplification which contained all mCpHs, unlike the lambda gDNA template. BS-Seq without copy-strand synthesis was not filtered. For DM-Seq, the copy strand does not amplify because the copy primer 5mCs are deaminated to Ts by A3A. DM-Seq filtering additionally eliminates dsDNA hairpins which can cause A3A non-deamination, similar to previously described enzymatic deamination protocols. Only reads with MAPQ≥30 were analyzed.

### Solid-Phase ACE and EM-Seq:

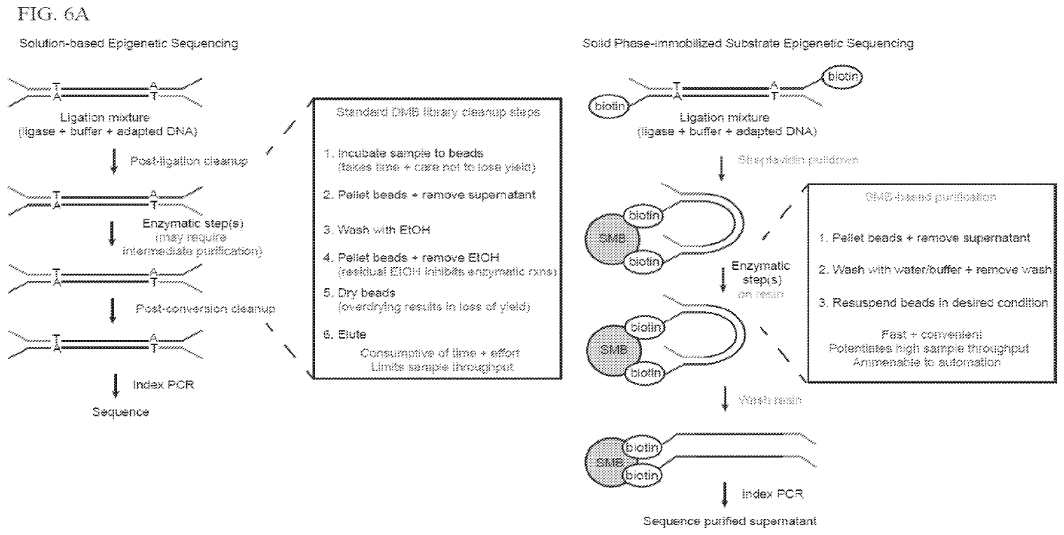

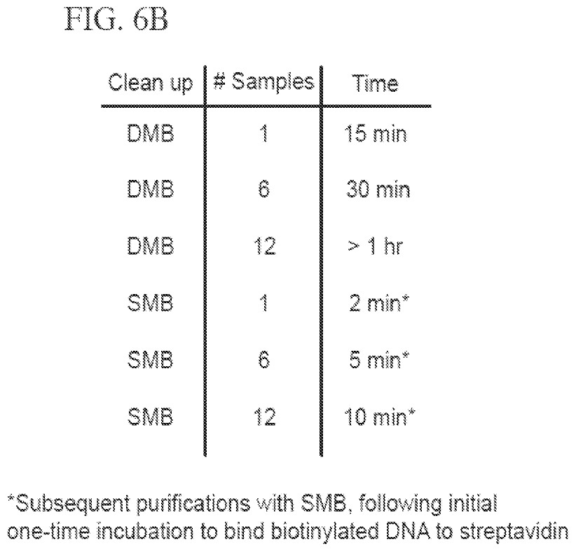

Sequencing pipelines were assessed for the viability of enzymatic steps occurring on immobilized DNA with modified adapters (see FIGS. 6 and 7). A mixture of CpG methylated pUC19, unmodified lambda gDNA, and fully 5hmC-modified T4 phage gDNA from a mutant lacking α/β glucosyltransferase enzymes were used as control input DNA. The DNA mixture was then subjected to the EM-Seq kit with the following modifications 1: instead of 5mC modified adapters provided in the kit, A3A-resistant adapters were used. 2: following adapter ligation, TdT was used to introduce biotin handles on the 3′ end of the adapted DNA. 3. In some conditions, biotinylated material was then fixed on streptavidin magnetic beads (SMB) and carried forward. 4. Enzymatic steps were performed either on immobilized substrates or in solution as noted by the table in FIG. 7B. Following library preparation, libraries were quantified by Qubit, quality checked by BioAnalyzer, sequenced on an Illumina MiSeq (150 bp paired end reads), and analyzed for deamination efficiency. For solid-phase ACE-Seq, the same procedure was followed with the omission of the TET oxidation step. Bioinformatic analysis was performed as described above.

Pre-Adapter bACE-Seq:

The viability of the bACE-Seq pipeline with modified engineered adapters was assessed (See FIG. 8A-C). A mixture of CpG methylated pUC19, unmodified lambda gDNA, and fully 5hmC-modified T4 phage gDNA from a mutant lacking α/β glucosyltransferase enzymes was used as control input DNA. The DNA mixture was sheared, end-repaired, and ligated to BS/A3A resistant adapters (e.g., 5hmC+βGT and 5pyrC). The mix was then purified using SPRI beads (1.2×) subjected to BS conversion (Diagenode) and split where part of the sample underwent subsequent A3A deamination. The resulting libraries were then indexed, quality checked via Qubit and BioAnalyzer, and sequenced on an Illumina MiSeq to determine conversion efficiencies.

### Multiplexed BS/A3A Experiment:

The viability of multiplexed bACE-Seq pipeline with modified engineered adapters was assessed (See FIG. 8E-F). A mixture of CpG methylated pUC19, unmodified lambda gDNA, and fully 5hmC-modified T4 phage gDNA from a mutant lacking α/β glucosyltransferase enzymes was used as control input DNA. Fully methylated Jurkat cell genomic DNA was also employed in this pipeline (see FIG. 8F). The DNA mixture was sheared, end-repaired, and ligated to BS/A3A-resistant adapters. Following adapter ligation, the adapted material was treated with TdT and biotin-16-ddUTP to introduce a biotin handle. The mix was then purified using SPRI beads (1.2×) and subjected to BS conversion (Diagenode). Following BS, the sample DNA was incubated and bound to SMB. The immobilized substrate was then used to generate a BS library by performing an indexing reaction on the immobilized substrate. The DNA substrate, still immobilized, was then taken through A3A deamination, and then indexed on-resin. Both libraries generated were quality checked via Qubit and BioAnalyzer and sequenced on an Illumina MiSeq instrument. To look for identical molecules present in both libraries, a script was written and applied to identify samples with the same starting 5′ end. Samples were visualized with integrated genome viewer (IGV) (FIG. 8F).

The following examples are provided to illustrate certain embodiments of the invention. They are not intended to limit the invention in any way.

### Example I

### Modified Cytosine Bases in Adapters are Resistant to Enzymatic Deamination

As shown in FIG. 2, natural cytosine variants are not compatible with enzymatic deamination, while bulky modifications to the 5-position make the cytosine resistant to enzymatic deamination. These resistant cytosines can be built into DNA molecules that can be ligated to target DNA samples in the form of adapters. The sequences of a few representative adapters compatible with Illumina next-generation sequencing are shown (FIG. 2B), where the X modification involved the modified cytosine base. These oligonucleotides can also be modified by a binding partner to allow for immobilization of the adapted DNA. The modifications for immobilization can be added off a nucleobase or at the ends of the oligonucleotide during synthesis or enzymatically after DNA synthesis (FIG. 2C).

### Modified Adapters Enable Pre-Deamination Library Preparation.

FIG. 3 relates to steps for preparation of a library comprising DNA for epigenetic sequencing analysis. FIG. 3A shows a post-deamination library preparation which have typically been necessary to avoid transformation of adapter sequences which must be preserved for proper loading onto a sequencer. This post-deamination strategy is costly in terms of both resources and time. FIG. 3B depicts a pre-deamination library preparation where adapters are ligated immediately following shearing and adapted material is then subjected to enzymatic deamination and carried through library preparation. In addition to streamlining the workflow, the pre-adapter strategy, made possible by modified adapters, opens up new abilities for enzymatic sequencing approaches for profiling multiple DNA modifications on the same DNA strand or simultaneous reading of genetic and epigenetic information, data which cannot be obtained in enzymatic pipelines with DNA deaminase-sensitive cytosine analogs.

To evaluate and identify if the proposed candidates can make DNA deaminase-based sequencing pipeline possible, lambda genomic DNA was sheared and ligated with adapters containing either unmodified C, 5mC, 5pyC, 5hmC, 5hmC+βGT, or 5pyrC modifications, with the later set as representative examples of adapters with analogs that might be resistant to enzymatic deamination. The adapted DNA was then subjected to either no treatment or enzymatic deamination by A3A. Library generation was attempted using the adapters as the priming site for PCR. When the different adapted samples were untreated and amplification was quantified by qPCR, they all took the same number of cycles to reach the specified threshold (CT) thereby indicating equivalent ability to be ligated. Following A3A deamination and qPCR amplification with primers binding to the adapter regions, the CT values for C and 5mC were in great excess of those for A3A-resistant analogs supporting that they are not suitable for pre-deamination workflows whereas 5pyC, 5hmC, 5hmC+βGT, and 5pyrC adapters amplified with efficiency demonstrating their appropriateness for a pre-deamination workflow. These examples support the use of modified adapters in solution phase-based sequencing pipelines, which are not able to be performed with currently used adapters containing unmodified cytosine or 5mC. See FIG. 3C.

### Example II

Enzymatic Deamination of Immobilized DNA Ligated with Modified Adapters

Deamination on DNA immobilized on a solid phase is especially attractive to pursue, as these workflows are streamlined in terms of time and yield and are also amenable to automation. Importantly, immobilized DNA can permit washing between steps in a protocol without the loss of DNA. Currently, many enzymatic sequencing pipelines with DNA deaminases require the use of user error-prone “snap cooling” protocols, as previously described in our extended methods manuscript in order to generate single-stranded DNA [45]. As an alternative to these snap cooling conditions, we wondered whether a solid phase, such as an avidin-containing magnetic bead, could be used to immobilize gDNA and leveraged as a platform on which A3A could act (FIG. 4). The ability for the enzyme to act upon immobilized DNA was a significant unknown and would open sequencing pipelines to several example applications shown here including repeated interrogation of the same DNA molecule more than once.

In this experiment, a homogenous PCR product was ligated to a forward strand adapter (red) and reverse strand adapter (blue) containing a 3′ biotin synthesized by solid-phase synthesis. These adapters at this stage did not contain DNA modifications to the cytosine base (unmodified C only) as the goal was to determine if DNA deaminase can act on immobilized DNA or not. We then bound the DNA to streptavidin resin. After subjecting the DNA to either an NaOH or wash buffer-only wash, the gDNA was then incubated at 37° C. for 1 hour with APOBEC3A, while still bound to the resin (FIG. 4A). After PCR amplification utilizing internal primers which amplify only the black region depicted, Sanger sequencing of the PCR product shows that all 27/27 cytosines were deaminated and sequenced as Ts. A ˜20 base pair window containing non-preferred −1 G and A was visualized by EditR analysis and shown here (FIG. 4B). The finding that <2% of cytosines are being called as Cs after NaOH wash enabled by resin-based deamination (red box) was especially promising because it includes purine (G and A) −1 sequence contexts which have previously been shown to be unfavorable for A3A deamination [16]

To next move to modified adapters and test a more complicated substrate with putative secondary structures that could inhibit A3A deamination, we treated 5pyC-adapter ligated lambda gDNA substrate to the enzyme terminal transferase (TdT) and incubated with biotin-ddUTP (16 linker) to tag the 3′-end. We subsequently attempted resin-based enzymatic deamination again, including a positive control snap cooling deamination condition (condition 1) and negative control condition with no NaOH wash (condition 6) as well as 4 experimental conditions with varying washing protocols (conditions 2-5). Notably, condition 2 shows an example of a wash protocol that decreases deamination efficiency. We subsequently amplified gDNA at a locus within lambda gDNA again and subjected the amplicon to interrogation of a single TCGA Taq^{α}I digestion site (FIG. 4C). These results studying a complex gDNA substrate qualitatively show that there are no deamination differences between a snap cooling positive control and enzymatic deamination on resin (conditions 3, 4, 5). An immobilized DNA-based enzymatic sequencing approach thus opens up multiple pipelines for epigenetic sequencing applications, especially when considering that multiple rounds of deamination can be performed between wash steps.

### Example III

### Solution-Phase Deamination of DNA Using Modified Adapters for Sequencing of 5mC

Modified adapters are also useful for enzymatic sequencing approaches taking place in solution, which would not be possible without adapters that are resistant to enzymatic deamination. An example of such a sequencing pipeline is provided by direct methylation sequencing (DM-Seq), which aims to directly detect 5mC alone by a C-to-T transition in sequencing and uses an engineered DNA methyltransferase that has taken on neomorphic DNA carboxymethyltransferase activity [13].

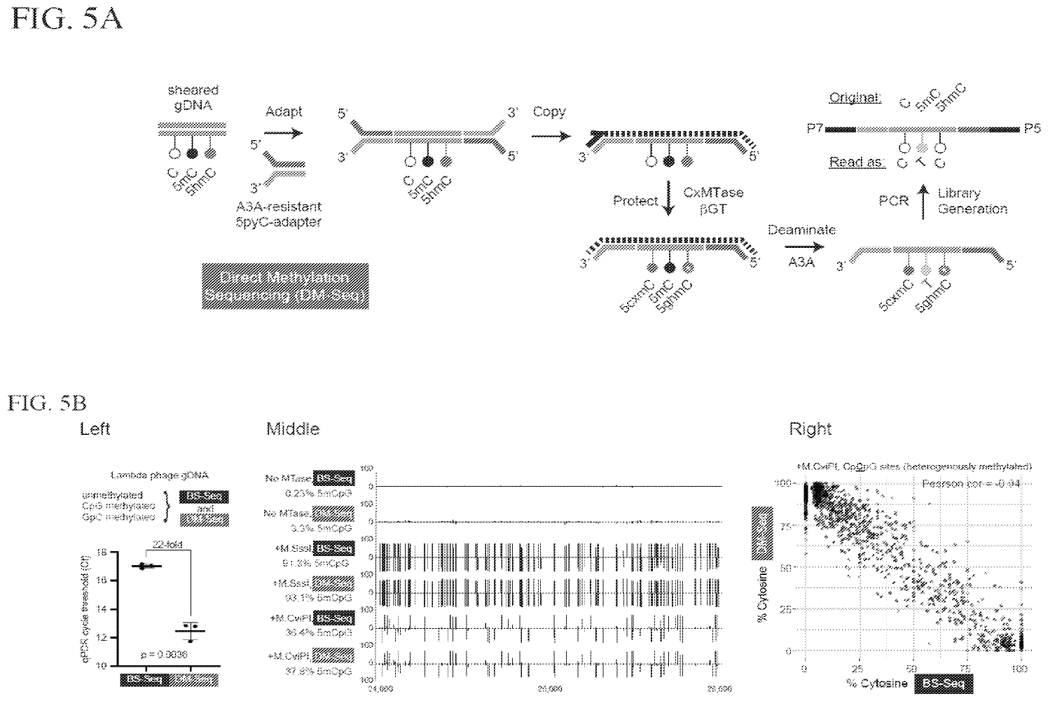

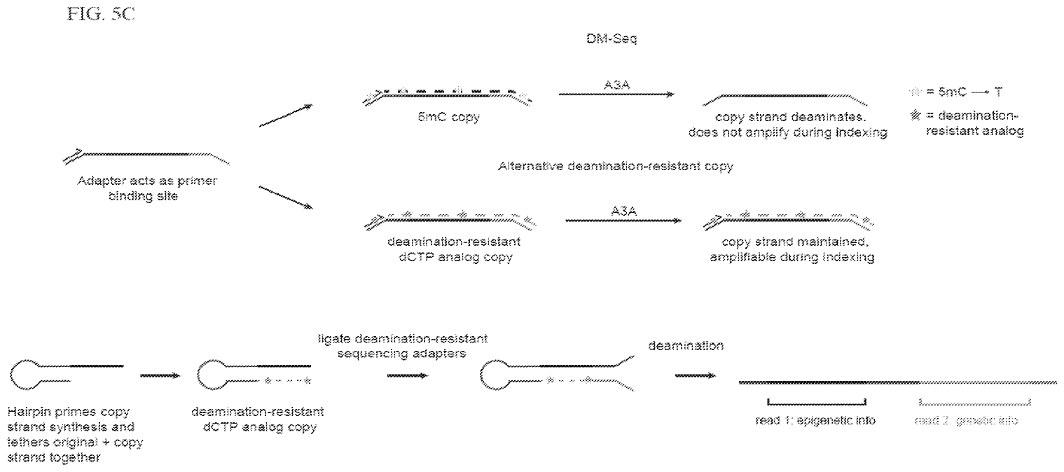

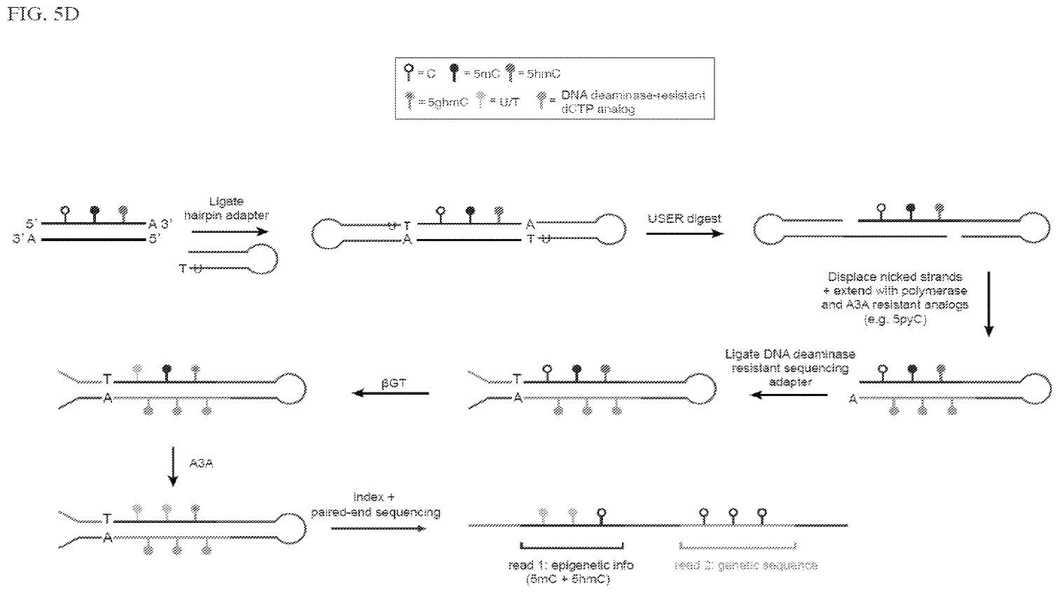

In the DM-Seq workflow (FIG. 5A), 5pyC adapters are ligated to sheared genomic DNA (gDNA). The adapter is then used to prime DNA synthesis with a DNA polymerase to create a strand exclusively containing 5mCs in place of C. The gDNA is then protected by the action of the CxMTase (on unmodified CpGs) and glucosylation by βGT (for 5hmCs). Subsequent deamination by A3A is performed before PCR amplification and sequencing. To quantify the fidelity of this workflow, we used three lambda phage gDNA samples: native gDNA as a standard with unmodified CpGs, gDNA methylated at CpG sites with M.SssI, and gDNA methylated at GpC sites with the MTase M.CviPI. Given GpC targeting, we anticipated that M.CviPI would provide heterogeneous levels of methylation at CpG sites throughout the genome. Sheared gDNA samples were split and then either ligated to 5mC-containing adapters and subjected to BS-Seq or ligated to 5pyC-containing adapters and processed by DM-Seq.

We first quantified the efficiency of library generation from the samples. Amplifiable DNA content post-deamination was 22-fold more across DM-Seq samples as compared to BS-Seq by qPCR (avg Ct=17.0 vs 12.5, FIG. 5B, left). We next focused on comparing the genome-wide efficiency of CxMTase protection and A3A-mediated deamination (FIG. 5B, middle). For the unmodified CpGs, we found a low rate of non-conversion by BS-Seq (0.23%), and a high rate of protection from deamination with DM-Seq (96.7%), validating the efficiency of the copy-strand protocol for CpG conversion to 5cxmCpG. For the gDNA sample treated with M.SssI, 91.3% of CpGs were protected from deamination with BS-Seq, with a comparable level (93.1%) deaminated by A3A in DM-Seq. In the M.CviPI MTase condition, we detected 95.4% of GpCpGs as methylated by BS-Seq and 94.5% as methylated by DM-Seq, while control WpCpGs (W=A/T) showed 2.8% and 5.2%, respectively. M.CviPI-treated gDNA provided an added opportunity to compare heterogeneous methylation, as this enzyme is known to have off-target activity at CpCpG sites. Across these sites, average methylation is similar: 29.3% and 31.4% for BS-Seq and DM-Seq, respectively. Importantly, when analyzed at the individual CpG level, the detection of 5mC is highly correlated (Pearson coefficient=−0.94 in CpCpGs, FIG. 5B, right). To our knowledge, correlations on matched, in vitro-generated, heterogeneously methylated samples such as M.CviPI-treated gDNA have not been benchmarked before. This experiment offers stronger validation relative to prior methods that attempt correlations across non-matched biological samples containing multiple confounding cytosine modifications and demonstrates the application of modified adapters containing unnatural DNA deaminase-resistant modifications to a DNA deaminase-based sequencing pipeline.

In DM-Seq, the 5mC copy strand is synthesized to increase CxMTase activity on CpG sites opposite the copy strand. Critically, this 5mC copy strand does not show up as sequencing reads as subsequent deamination by A3A prevents downstream amplification. If instead the copy strand step is performed with A3A-resistant dCTP analogs such as the cytosine bases shown in FIG. 2A, the copy strand persists through library preparation and sequencing (FIG. 5C, Top). In such an approach, the library would then contain molecules that contain the epigenetic information, with deaminated cytosines, and molecules that contain the starting genetic information. These strands could be matched by their shared 5′ and 3′ ends or using UMIs.

### Example IV

Simultaneous Epigenetic and Genetic Analysis Using Modified Adapters and Copying of DNA with DNA Deaminase-Resistant Cytosine Analogs

Reading the epigenetic code requires reactivity of DNA with reagents that selectively deaminate or alter the readout of different modification states of cytosine. These methods for deamination act on both Watson and Crick strands of DNA, most commonly deaminating all unmodified cytosines. This results in the limitation of reduced mapping efficiency and ability to error correct for sequencing read errors as unmodified cytosine, one of the four units of code of DNA, transitions to thymine and thus the genetic code is reduced from four bases to three.

Taking inspiration from hairpin bisulfite approaches, we realized that our discovery of DNA deaminase resistant cytosine analogs could be leveraged for the simultaneous analysis of genetic and epigenetic information. Notably, while such approaches have been applied for bisulfite before, these precedents would not work for DNA deaminase-based enzymatic sequencing workflows, as the 5mC bases used in bisulfite-based methods are deaminated by DNA deaminases like APOBEC3A. In our modified workflow, a top strand of interest is linked to a copy strand that contains the DNA deaminase-resistant cytosines. As the original target strand and deamination-resistant copy strand are linked, sequencing both halves of the molecule generates the genetic and epigenetic information together (FIG. 5C Bottom). A schematic is provided with one method for achieving this goal (FIG. 5D). Here, the standard initial library preparation steps of shearing sample DNA and end-repairing to generate single A-tail overhangs could be used to add on uracil-containing hairpin linkers to both ends. The presence of these uracil bases within the hairpins allows for site-specific cleavage by treatment with UDG and endonuclease (ex. USER Enzyme). The nicks introduced provide means to separate the hairpin-adapted strands into two single strands that each contain a single hairpin on one end. A polymerase coupled with a dNTP mix where dCTP is substituted with A3A-resistant analogs can then be used to generate a copy strand that exclusively contains A3A-resistant C analogs. Subsequent A-tailing of the blunt-ended molecule generated can then allow for ligation of adapters containing the same or different A3A-resistant bases. These molecules can have native 5hmC's protected by βGT and then be deaminated by A3A. Following indexing, the libraries can then be sequenced in paired-end mode to have both genetic and epigenetic information read out (FIG. 5D). Thus, the protocol follows logically from the success of direct methylation sequencing (FIG. 5), with the key differences being the presence of a hairpin adapter to start strand copying and the use of a DNA deaminase-resistant cytosine analog in lieu of 5mC, which is DNA deaminase-susceptible.

A strength of the methods where the genetic information is tethered to the epigenetic information in the same read is that these reads can be enriched using probe oligonucleotides that are complementary to the DNA regions of interest. The present approach provides certain advantages over prior art, wherein probes are unable to reliably isolate and enrich samples when the genetic information is lost by deamination.

### Example V

Epigenetic Sequencing of 5hmC and 5mC with Solid-Phase Immobilized Substrates

If enzyme activities that alter the readout of these bases, beyond enzymatic DNA deamination, were also compatible with immobilized DNA, the epigenetic bases that can be detected via solid phase-based sequencing workflows would be greatly expanded. Two enzymes that are commonly used for epigenetic sequencing are β-glucosyltransferase (β-GT) which glucosylates and prevents low-level 5hmC deamination by A3A and TET enzymes which iteratively oxidize 5mC to 5caC thus protecting 5mC from A3A deamination and allowing for the simultaneous detection of 5mC and 5hmC. In ACE-Seq developed by our laboratory, 5hmC in DNA is modified by glucosylation and then then C and 5mC are deaminated by A3A. In EM-Seq, a method that was developed after ACE-Seq, 5mC is oxidized by TET enzymes with simultaneous treatment with β-GT to convert 5mC and 5hmC to a mixture of glucosylated 5hmC and 5caC, both of which are resistant to A3A-mediated deamination.

Current methods for ACE and EM-Seq require that they take place on solution-based substrates. That substrates are free in solution provides an added layer of complication for moving between enzymatic steps. To facilitate these different enzymatic steps, enzymes from earlier steps and associated buffers much be purified away and then exchanged. The standard is to use either columns that bind to DNA reversibly or solid-phase reversible immobilization (SPRI) methods with DNA-binding magnetic beads (DMB) that reversibly bind DNA non-specifically. Notably, such reversible binding is not compatible with the enzymatic workflows on solid phase that we explore in this document. Purification steps commonly follow every enzymatic step of the sequencing pipeline and require excessive handling and time, thus also limiting the number of samples that can be processed by individuals (FIG. 6A Left). In comparison, following a single incubation event with streptavidin magnetic beads (SMB), DNA substrates that have been adapted with biotinylated adapters can be easily manipulated through the same workflow using SMB and a magnetic rack (FIG. 6A Right). Analogous pathways could be utilized with different binding partners on the DNA adapter and on the solid phase. SMB pulldown is rapid, allowing for a more efficient exchange of buffer that negates the need for incubation at each step as required by DMB and is simpler to perform without the need for ethanol (EtOH)-based washes which can either inhibit subsequent enzymatic reactions or lower yield. A comparison of the time it takes to process samples with either SMB or DMB is provided (FIG. 6B).

To evaluate if, like A3A, the action of these two enzymes coupled with deamination by A3A could also be performed on immobilized substrate, we compared enzymatic epigenetic sequencing methods with both solution-based substrates and solid-phase immobilized substrates (workflows presented in FIG. 7A). To rigorously determine deamination efficiencies, three substrates pooled together were used: unmethylated lambda DNA (acting as a C control), methylated pUC19 (acting as an 5mC control), and T4-5hmC genomic DNA (acting as a 5hmC control). This later samples involved a mutant version of the T4 phage that lack the glucosyltransferase enzymes, and is thus entirely populated with 5hmC in lieu of unmodified C. In this experiment, the pooled DNA samples were subjected to either the published ACE-Seq and EM-Seq protocols or the standard protocols altered to accommodate immobilized DNA substrate. A notable modification for all workflows evaluated being that A3A-resistant adapters were used. The other notable changes to the published protocols being that following adapter ligation, adapted DNA was biotinylated with TdT and biotin-ddUTP. For non-solution-based comparator samples, substrates were bound to streptavidin magnetic beads (SMB). Enzymatic steps were carried out either on substrates free in solution or on immobilized DNA substrates (conditions noted in FIG. 7B). For SMB-bound substrates, wash steps and buffer exchanges were performed on resin, replacing SPRI purification steps.

Promisingly, the readout of each control DNA for each sample was in line with expectation where ACE-Seq (both solution and solid-phase immobilized) discriminated 5hmC from C and 5mC containing substrates and EM-Seq (both solution and solid-phase immobilized) discriminated 5hmC+5mC from C containing substrates (FIG. 7B). The fact that all combinations of solid-phase-based and solution-based enzymatic steps yielded nearly identical deamination efficiencies supports that both R-GT and TET enzymes efficiently act on solid-phase immobilized DNA substrates, thus permitting the generation of solid phase ACE-Seq (spACE-Seq) and solid-phase immobilized EM-Seq, also termed by us as resin EM-Seq (rEM-Seq). The development of these solid-phase-immobilized epigenetic sequencing methods has the potential to offer several notable advantages including the simplification of workflows and the greater retention of input DNA. Because of the number of purification steps required for these enzymatic pipelines, replacement of each DMB step with SMB step provides a significant time saving and greatly increases the number of samples that can be processed by individuals without the need for specialized liquid handling robots. Excitingly, the ability to retain immobilized DNA substrate through the entire workflow enables rapid switching between enzymatic conditions without the need to transfer sample between tubes for purification. Thus, this process is highly amenable to automation where following adapter ligation, samples could be immobilized by SMB and different reaction conditions could be either robotically added and removed or flowed over analogous popular solid phase coupled synthesis methods used for generation of peptides and oligos. Alternatively, rather than requiring a bead-based resin (eg. SMB) where the bead is pulled down, the method could be accomplished with any container serving as the solid support (including without limitation, a vessel, a test tube, a multi-well plate) where the surface of said container is coated in a specific binding partner (e.g. multi-well PCR plate coated with streptavidin or PCR tubes coated with streptavidin). In this scheme, following adapter ligation of the target DNA, the adapted target DNA can be directly immobilized to the container (e.g. well or tube) itself and the reaction conditions can be directly added to or removed from the container. This confers numerous advantages to both automated and non-automated workflows as it removes the need for a magnetic rack and bead reagents, and it eliminates both the time required to pellet the beads and resuspend them in solution and the risk of disturbing the pelleted beads which could reduce yield.

### Example VI

Epigenetic Sequencing with Chemical/Enzymatic Deamination Resistant Adapters and Reiterative Interrogation of the Same DNA Molecule in Library Constructs for Resolving 5mC and 5hmC.

Workflows that couple chemical and enzymatic methods of deamination could also greatly benefit from a pre-deamination adapter strategy. An example is a method our group developed termed bACE-Seq which results in two libraries: a standard BS library and a post-A3A library where 5mC is also deaminated (FIG. 8A). The comparison of the two libraries allows for separate detection of 5mC+5hmC versus 5hmC alone. To determine if our adapter candidates were also resistant to bisulfite, we subjected them to an experiment analogous to the one presented in Example I. Here, following ligation of the adapters to sheared lambda gDNA, the samples were subjected to BS treatment and then amplification was quantified by qPCR using primers that bind the adapter region (FIG. 8B). This experiment revealed that candidates 5hmC, 5hmC+βGT, and 5pyrC adapters all demonstrate resistance to BS, providing examples of the overall strategy being pursued with dual bisulfite and enzymatic resistant adapters.

Promising adapters were then used to pilot bACE-Seq using a pre-deamination adapter ligation strategy. Deamination efficiencies on control DNA from libraries prepared with this strategy are provided demonstrating the viability of this strategy (FIG. 8C). As demonstrated in the bisulfite libraries, the conversion efficiencies fall in line with expectation as deamination of C is observed, but not 5mC and 5hmC. After the A3A deamination step is carried out, the bACE-Seq library is generated, demonstrating that the adapters tolerated both bisulfite and A3A deamination. In the resulting library reads, the 5mC bases are now deaminated, showing how discrimination of 5mC from 5hmC could take place in libraries.

A never-before demonstrated advantage of the solid-phase-immobilized deamination method is that the same DNA molecule can be interrogated more than once in library constructs. For example, DNA that has been treated with bisulfite leads to the conversion of C to U. 5mC is resistant to deamination, while 5hmC is converted to the adduct CMS. If this bisulfite-converted DNA is then enzymatically deaminated using A3A, the 5mC will convert to T, but the 5hmC (protected as CMS) will not. Deamination of solid-phase-immobilized substrates could optionally be partnered with either barcodes on the adapters (a string of 8 random (N) nucleotides that serves as a molecular barcode also referred to as an MID) or a decoding strategy using the unique 5′ and 3′ ends generated from shearing, the latter of which we demonstrate in this example. A library could be generated from the immobilized DNA after bisulfite and then again after A3A. The comparison of either molecule's start and end position or the barcodes could then be used the decode when 5mC and 5hmC are present on the original starting DNA molecule. The generation of two libraries from the same starting DNA is a distinctive potential advantage of deamination protocols performed on immobilized DNA. To parse the status of C, 5mC, and 5hmC in cis, companion bioinformatic tools must be developed which underlie this method. A schematic representing one way that this could be achieved is presented (FIG. 8E).

To demonstrate the power of this approach and in pilot experiments, we have found that BS and bACE libraries generated using immobilized substrates result in overlapping reads which can be used to determine the modification status of insert. An example of the same molecule being read twice, once following BS and the second following A3A is provided (FIG. 8F). In this figure, we demonstrate using Jurkat T cell genomic DNA that was fully methylated at CpGs that the same molecule can be pulled out from sequencing library one and two. After library one, the CpG site is shown as modified, which can be either 5mC or 5hmC. The second library shows that this site is deaminated which means that it can be definitively assigned as being 5mC and not 5hmC. When applied to a molecule that contains both 5mC and 5hmC in the same starting DNA molecule, this iterative assessment of methylation status can definitively parse 5mC and 5hmC in the same DNA molecule. To our knowledge, this also represents the first time an epigenetic sequencing library is generated from the same starting DNA molecule more than once with differential cytosine modification states revealed in each stage.

Precedents from the above method for parsing the status of C, 5mC, and 5hmC in cis (in the same strand) and the above method for retention of genetic information in a single molecule (FIG. 5D) could be combined to generate a single method for parsing C, 5mC, and 5hmC while also maintaining the original four-letter code of DNA. A representative schematic is provided for achieving this dual read of the ternary epigenetic code (C, 5mC, 5hmC) with simultaneous genetic code. In this representative workflow, sample DNA is sheared and ligated to hairpin adapters. Separation of the strands, as noted above, allow the hairpins to prime a copy step where BS/A3A-resistant cytosine analogs (e.g., 5hmC+βGT) can be incorporated. Following generation of the copy strand with the resistant analogs and A-tailing, sequencing adapters containing these BS/A3A-resistant analogs and a biotin handle can be ligated. At this stage, the same strategies used directly above for multiplexing BS/bACE readouts can be applied where the molecules are BS-treated, bound to SMB, indexed with one set of indexing primers, A3A-treated, and then indexed with a separate set of indexing primers. The indexed libraries can then be sequenced out (FIG. 8G) to reveal differential epigenetic states in Read 1, with the intact, non-deaminated genetic code in Read 2. A strength of the methods where the genetic information is tethered to the epigenetic information in the same read, is that these reads can be enriched using probe oligonucleotides that are complementary to the DNA regions of interest. Such probes are unable to reliably isolate and enrich samples when the genetic information is lost by deamination.

### Example VII

Analysis of Circulating Cell Free DNA (cfDNA)

Together, the C/5mC/5hmC distribution at CpGs provides a molecular fingerprint primed for application to cancer diagnostics. In one approach, with high-input cfDNA quantities (>250 ng), tissue-specific differentially methylated regions (DMRs) were used to determine the relative contribution of tissues to cfDNA in cancers. Affinity-capture or immunoprecipitation (IP) techniques (FIG. 1B) have also recently been applied to isolate 5mC- or 5hmC-containing cfDNA to aid in tumor diagnostics; however, enriching for 5mC- or 5hmC-marked cfDNA fails to provide any information about where those marks are specifically located in the sequenced DNA. For base-resolution epigenetics, the current gold standards depend on bisulfite-based (BS-Seq) approaches. BS-Seq relies upon the differential susceptibility of modified cytosine bases to chemical deamination with sodium bisulfite. Unmodified cytosine bases are readily deaminated, while modified cytosines are resistant. As noted above, BS-based approaches suffer from two major hurdles that constrain their widespread adoption to cfDNA analysis (FIG. 1A): (1) bisulfite itself is unable to distinguish between 5mC and 5hmC and (2) harsh chemical deamination is highly destructive, typically degrading >99% of input DNA, which particularly impedes the study of sparse cfDNA.

Enzymatic deamination approaches, such as used in ACE-Seq, can overcome some of the limitations imposed by bisulfite. However, enzymatic approaches also have two challenges that are notable:

First, the current strategy for using adapters is not compatible for DNA deamination alone. In processing of DNA samples, the most common approach involves taking sheared DNA (or naturally sheared DNA in the case of cfDNA) and placing on terminal adapters that can be used to generate sequencing libraries. These adapters commonly used 5mC in place of unmodified C, as this base is resistant to bisulfite; however, DNA deaminases of the AID/APOBEC family lead to the deamination of 5mC, which means that these adapters are not compatible for library generation. Thus, we hypothesized that the ideal set of adapters would be ones resistant to enzymatic deamination and also resistant to bisulfite-mediated deamination as described in Example I.

Second, for all sequencing pipelines, between each step, the DNA is typically washed and/or purified, in order to prepare it for subsequent steps in the sequencing pipeline. With each purification step there is a loss of DNA which means that the final libraries generated do not represent the full diversity present in the initial population of the sample. This problem is particularly acute with regards to sparse samples such as cfDNA, where preserving DNA is important.

Separate from the two issues above, all currently employed methods only permit one to generate a single library from a single starting template DNA molecule. Notably, the compositions and methods described herein enable generation of a library at different interval steps along the sequencing pipeline, thereby making it possible to interrogate the same DNA molecule more than once to, for example, parse 5hmC from 5mC, as we have demonstrated in FIG. 8F.

Lastly, we have noted that with the use of adapters resistant to DNA deaminases and with strand copying with DNA deaminase resistant dCTPs, genetic information can be tethered to epigenetic information in the same read. This approach also means these reads can be enriched using probe oligonucleotides that are complementary to the DNA regions of interest, a process which is particularly important for cfDNA where there are probes of high value to diagnostics.

The modified adapter strategy that is tolerant to enzymatic deamination and permits enzymatic DNA deamination on an immobilized DNA substrate can be used to advantage to interrogate methylated DNA molecules from a variety of biological sources.

### REFERENCES

[1] Hesson, L. B., Pritchard, A. L., 2019. Clinical Epigenetics. 1st ed: Springer.
[2] Hotchkiss, R. D., 1948. The quantitative separation of purines, pyrimidines, and nucleosides by paper chromatography. Journal of Biological Chemistry 175:315-332.
[3] Wilson, G. G., Murray, N. E., 1991. Restriction and Modification Systems. Annual Review of Genetics 25:585-627.
[4] Schubeler, D., 2015. Function and information content of DNA methylation. Nature 517:321-326.
[5] Nabel, C. S., Manning, S. A., Kohli, R. M., 2011. The Curious Chemical Biology of Cytosine: Deamination, Methylation, and Oxidation as Modulators of Genomic Potential. ACS chemical biology.
[6] Bird, A. P., Southern, E. M., 1978. Use of restriction enzymes to study eukaryotic DNA methylation: I. The methylation pattern in ribosomal DNA from Xenopus laevis. Journal of Molecular Biology 118:27-47.
[7] Frommer, M., McDonald, L. E., Millar, D. S., Collis, C. M., Watt, F., Grigg, G. W., et al., 1992. A genomic sequencing protocol that yields a positive display of 5-methylcytosine residues in individual DNA strands. Proceedings of the National Academy of Sciences of the United States of America 89:1827-1831.
[8] Tahiliani, M., Koh, K. P., Shen, Y., Pastor, W. A., Bandukwala, H., Brudno, Y., et al., 2009. Conversion of 5-methylcytosine to 5-hydroxymethylcytosine in mammalian DNA by MLL partner TET1. Science (New York, N.Y.) 324:930-935.
[9] Ito, S., Shen, L., Dai, Q., Wu, S. C., Collins, L. B., Swenberg, J. A., et al., 2011. Tet proteins can convert 5-methylcytosine to 5-formylcytosine and 5-carboxylcytosine. Science (New York, N.Y.) 333:1300-1303.
[10] He, Y. F., Li, B. Z., Li, Z., Liu, P., Wang, Y., Tang, Q., et al., 2011. Tet-mediated formation of 5-carboxylcytosine and its excision by TDG in mammalian DNA. Science (New York, N.Y.) 333:1303-1307.
[11] Kriaucionis, S., Heintz, N., 2009. The nuclear DNA base 5-hydroxymethylcytosine is present in Purkinje neurons and the brain. Science (New York, N.Y.) 324:929-930.
[12] Huang, Y., Pastor, W. A., Shen, Y., Tahiliani, M., Liu, D. R., Rao, A., 2010. The Behaviour of 5-Hydroxymethylcytosine in Bisulfite Sequencing. PLoS ONE 5:e8888.
[13] Wang, T., Kohli, R. M., 2021. Discovery of an Unnatural DNA Modification Derived from a Natural Secondary Metabolite. Cell chemical biology 28:97-104.e4.
[14] Kohli, R. M., Zhang, Y., 2013. TET enzymes, TDG and the dynamics of DNA demethylation. Nature 502:472-479.
[15] Nabel, C. S., Jia, H., Ye, Y., Shen, L., Goldschmidt, H. L., Stivers, J. T., et al., 2012. AID/APOBEC deaminases disfavor modified cytosines implicated in DNA demethylation. Nature chemical biology 8:751-758.
[16] Schutsky, E. K., Nabel, C. S., Davis, A. K. F., DeNizio, J. E., Kohli, R. M., 2017. APOBEC3A efficiently deaminates methylated, but not TET-oxidized, cytosine bases in DNA. Nucleic acids research 45:7655-7665.
[17] Shi, K., Carpenter, M. A., Banerjee, S., Shaban, N. M., Kurahashi, K., Salamango, D. J., et al., 2017. Structural basis for targeted DNA cytosine deamination and mutagenesis by APOBEC3A and APOBEC3B. Nature Structural &amp; Molecular Biology 24:131.
[18] Schutsky, E. K., DeNizio, J. E., Hu, P., Liu, M. Y., Nabel, C. S., Fabyanic, E. B., et al., 2018. Nondestructive, base-resolution sequencing of 5-hydroxymethylcytosine using a DNA deaminase. Nat. Biotech. 36:1083-1090.
[19] Vaisvila, R., Ponnaluri, V. K. C., Sun, Z., Langhorst, B. W., Saleh, L., Guan, S., et al., 2021. Enzymatic methyl sequencing detects DNA methylation at single-base resolution from picograms of DNA. Genome research 31:1280-1289.
[20] Sun, Z., Vaisvila, R., Hussong, L. M., Yan, B., Baum, C., Saleh, L., et al., 2021. Nondestructive enzymatic deamination enables single-molecule long-read amplicon sequencing for the determination of 5-methylcytosine and 5-hydroxymethylcytosine at single-base resolution. Genome research 31:291-300.
[21] Caldwell, B. A., Liu, M. Y., Prasasya, R. D., Wang, T., DeNizio, J. E., Leu, N. A., et al., 2021. Functionally distinct roles for TET-oxidized 5-methylcytosine bases in somatic reprogramming to pluripotency. Molecular cell 81:859-869.e8.
[22] Iyer, L. M., Zhang, D., Rogozin, I. B., Aravind, L., 2011. Evolution of the deaminase fold and multiple origins of eukaryotic editing and mutagenic nucleic acid deaminases from bacterial toxin systems. Nucleic acids research 39:9473-9497.
[23] Krishnan, A., Iyer, L. M., Holland, S. J., Boehm, T., Aravind, L., 2018. Diversification of AID/APOBEC-like deaminases in metazoa: multiplicity of clades and widespread roles in immunity. Proceedings of the National Academy of Sciences of the United States of America 115:E3201-E3210.
[24] Song, C., Szulwach, K. E., Fu, Y., Dai, Q., Yi, C., Li, X., et al., 2010. Selective chemical labeling reveals the genome-wide distribution of 5-hydroxymethylcytosine. Nature biotechnology:1-8.
[25] Han, D., Lu, X., Shih, A. H., Nie, J., You, Q., Xu, M. M., et al., 2016. A Highly Sensitive and Robust Method for Genome-wide 5hmC Profiling of Rare Cell Populations. Molecular cell 63:711-719.
[26] Gao, P., Lin, S., Cai, M., Zhu, Y., Song, Y., Sui, Y., et al., 2019. 5-Hydroxymethylcytosine profiling from genomic and cell-free DNA for colorectal cancers patients. Journal of Cellular and Molecular Medicine 23:3530-3537.
[27] Li, W., Zhang, X., Lu, X., You, L., Song, Y., Luo, Z., et al., 2017. 5-Hydroxymethylcytosine signatures in circulating cell-free DNA as diagnostic biomarkers for human cancers. Cell research 27:1243-1257.
[28] Song, C. X., Yin, S., Ma, L., Wheeler, A., Chen, Y., Zhang, Y., et al., 2017. 5-Hydroxymethylcytosine signatures in cell-free DNA provide information about tumor types and stages. Cell research 27:1231-1242.
[29] Hu, L., Liu, Y., Han, S., Yang, L., Cui, X., Gao, Y., et al., 2019. Jump-seq: Genome-Wide Capture and Amplification of 5-Hydroxymethylcytosine Sites. Journal of the American Chemical Society 141:8694.
[30] Gibas, P., Narmontė, M., Staševskij, Z., Gordevičius, J., Klimašauskas, S., Kriukienė, E., 2020. Precise genomic mapping of 5-hydroxymethylcytosine via covalent tether-directed sequencing. PLoS biology 18:e3000684.
[31] Iyer, L. M., Tahiliani, M., Rao, A., Aravind, L., 2009. Prediction of novel families of enzymes involved in oxidative and other complex modifications of bases in nucleic acids. Cell cycle (Georgetown, Tex.) 8:1698-1710.
[32] Yu, M., Hon, G. C., Szulwach, K. E., Song, C. X., Zhang, L., Kim, A., et al., 2012. Base-resolution analysis of 5-hydroxymethylcytosine in the mammalian genome. Cell 149:1368-1380.
[33] Yu, M., Hon, G. C., Szulwach, K. E., Song, C. X., Jin, P., Ren, B., et al., 2012. Tet-assisted bisulfite sequencing of 5-hydroxymethylcytosine. Nature protocols 7:2159-2170.
[34] Booth, M. J., Branco, M. R., Ficz, G., Oxley, D., Krueger, F., Reik, W., et al., 2012. Quantitative sequencing of 5-methylcytosine and 5-hydroxymethylcytosine at single-base resolution. Science 336:934-937.
[35] Liu, Y., Siejka-Zielinska, P., Velikova, G., Bi, Y., Yuan, F., Tomkova, M., et al., 2019. Bisulfite-free direct detection of 5-methylcytosine and 5-hydroxymethylcytosine at base resolution. Nature biotechnology 37:424-429.
[36] Liu, Y., Hu, Z., Cheng, J., Siejka-Zielinska, P., Chen, J., Inoue, M., et al., 2021. Subtraction-free and bisulfite-free specific sequencing of 5-methylcytosine and its oxidized derivatives at base resolution. Nature communications 12:618-2.
[37] Iyer, L. M., Abhiman, S., Aravind, L., 2011. Natural history of eukaryotic DNA methylation systems. Progress in molecular biology and translational science 101:25-104.
[38] Renbaum, P., Abrahamove, D., Fainsod, A., Wilson, G. G., Rottem, S., Razin, A., 1990. Cloning, characterization, and expression in Escherichia coli of the gene coding for the CpG DNA methylase from Spiroplasma sp. Strain MQ1 (M.SssI). Nucleic acids research 18:1145-1152.
[39] Wu, H., Wu, X., Shen, L., Zhang, Y., 2014. Single-base resolution analysis of active DNA demethylation using methylase-assisted bisulfite sequencing. Nature biotechnology 32:1231-1240.
[40] Dalhoff, C., Lukinavicius, G., Klimasauskas, S., Weinhold, E., 2006. Direct transfer of extended groups from synthetic cofactors by DNA methyltransferases. Nature chemical biology 2:31-32.
[41] Kriukienė, E., Labrie, V., Khare, T., Urbanavičiūtė, G., Lapinaitė, A., Koncevičius, K., et al., 2013. DNA unmethylome profiling by covalent capture of CpG sites. Nature communications 4:2190.
[42] Ličyte, J., Gibas, P., Skardžiūtė, K., Stankevičius, V., Rukšenaitė, A., Kriukienė, E., 2020. A Bisulfite-free Approach for Base-Resolution Analysis of Genomic 5-Carboxylcytosine. Cell reports 32:108155.
[43] Liang, J., Zhang, K., Yang, J., Li, X., Li, Q., Wang, Y., et al., 2021. A new approach to decode DNA methylome and genomic variants simultaneously from double strand bisulfite sequencing. Briefings in bioinformatics 22:bbab201. doi: 10.1093/bib/bbab201.
[44] Laird, C. D., Pleasant, N. D., Clark, A. D., Sneeden, J. L., Hassan, K. M., Manley, N. C., et al., 2004. Hairpin-bisulfite PCR: assessing epigenetic methylation patterns on complementary strands of individual DNA molecules. Proceedings of the National Academy of Sciences of the United States of America 101:204-209.
[45] Wang, T., Luo, M., Berrios, K. N., Schutsky, E. K., Wu, H., Kohli, R. M., 2021. Bisulfite-Free Sequencing of 5-Hydroxymethylcytosine with APOBEC-Coupled Epigenetic Sequencing (ACE-Seq). Methods in molecular biology (Clifton, N.J.) 2198:349-367.
[46] Kluesner, M. G., Nedveck, D. A., Lahr, W. S., Garbe, J. R., Abrahante, J. E., Webber, B. R., et al., 2018. EditR: A Method to Quantify Base Editing from Sanger Sequencing. The CRISPR journal 1:239-250.

While certain of the preferred embodiments of the present invention have been described and specifically exemplified above, it is not intended that the invention be limited to such embodiments. All patents, patent applications, and publications cited herein are expressly incorporated by reference in their entirety for all purposes. Various modifications may be made thereto without departing from the scope and spirit of the present invention, as set forth in the following claims.

1. An oligonucleotide adapter comprising a modified cytosine base resistant to enzymatic deamination, which confers deamination resistance in the cytosine bases selected from the group of 5-propynylC (5pyC), 5-pyrrolo-dC (5pyrC), 5-hydroxymethylcytosine (5hmC), glucosylated 5-hydroxymethylcytosine (5ghmC), cytosine 5-methylenesulfonate (CMS), N4-modified cytosine, and a bulky C5-position modified cytosine, wherein said oligonucleotide is optionally also resistant to chemical deamination.

2. The oligonucleotide of claim 1, wherein modification is 5pyC, 5pyrC, and 5hmC or a modified variant thereof.

3. The oligonucleotide of claim 1, operably linked to a first member of a specific binding pair, wherein said specific binding pair comprises first and second members selected from streptavidin-biotin, avidin-biotin, biotin analog-avidin, desthiobiotin-streptavidin, desthiobiotin-avidin, iminobiotin-streptavidin, iminobiotin-avidin, antigen-antibody, receptor-hormone, receptor-ligand, agonist-antagonist, lectin-carbohydrate, Fc receptor-mouse IgG-protein A, and virus-receptor binding pairs, said first and second binding pair members forming a specific-binding pair complex when brought together, wherein one member of said pair being operably linked to a magnetic particle or bead.

4.-6. (canceled)

7. A method for assessment of the methylation state of a DNA molecule via enzymatic or a combination of chemical and enzymatic deamination of an immobilized target DNA molecule, comprising
a) providing a nucleic acid sample comprising methylated DNA wherein the DNA is optionally sheared or is naturally between 50 to 1000 nucleotides in length;
b) conjugating the oligonucleotide adapter of claim 3 to the DNA of step a);
c) contacting the oligonucleotide of step b) with a solid support comprising the second member of said specific binding pair, thereby forming a duplex DNA containing specific binding member pair complex on a surface of said solid support;
d) incubating said duplex DNA containing specific binding pair complex under conditions which denature said duplex DNA, thereby producing single-stranded DNA;
e) contacting the single-stranded DNA containing specific binding member pair complex of step d) with at least one deaminase;
f) PCR amplifying the deaminase-treated DNA; and
g) sequencing PCR amplicons obtained from step f) and generating methylation profiles for said target DNA molecule.

8. The method of claim 7, wherein the DNA of step a) or step c) is treated with at least one glucosyltransferase, methyltransferase, polymerase, and/or TET enzyme, and the appropriate substrates thereof, a chemical agent for deamination, said agent being selected from bisulfite, pyridine borane, and borane-mediated deamination reagent.

9.-10. (canceled)

11. The method of claim 7, wherein said DNA in step a) or step c) is contacted with a glucosyltransferase and a UDP glucose derivative, thereby site specifically labeling all 5hmC bases with a glucose or modified glucose prior to performance of steps b)-g)
or
wherein said DNA in step a) or c) is contacted with at least one TET enzyme thereby catalyzing oxidation of 5mC to 5hmC, 5hmC to 5fC and 5fC to 5caC prior to performance of downstream steps
or
wherein said DNA in step a) or c) is contacted with a methyltransferase, thereby converting unmodified cytosines in the methyltransferase recognition sites on said DNA into 5-modified-cytosines.

12.-13. (canceled)

14. The method of claim 7, wherein said DNA in step b) or c) is copied by a polymerase with unmodified or non-deamination-resistant dCTP analogs to generate a copy strand of the target DNA that contains deamination-susceptible cytosines or
wherein said DNA in step b) or c) is copied by a polymerase with deamination-resistant dCTP analogs (e.g., 5pyC) to generate a copy strand of the target DNA that contains deamination-resistant cytosines
or
wherein said DNA in step b) or c) is copied by a polymerase which incorporates deamination-resistant dCTP analogs in a copy strand of the target DNA that contains deamination-resistant cytosines, and wherein the two strands of an original DNA strand and copy DNA strand are conjugated via an oligonucleotide adapter, which can be the same or different from the adapter of step b).

15.-16. (canceled)

17. A method for assessment of the methylation state of a DNA molecule via enzymatic or a combination of chemical and enzymatic deamination of a target DNA molecule in solution, comprising
a) providing a nucleic acid sample comprising methylated duplex DNA said DNA optionally being sheared or naturally between 50 to 1000 nucleotides in length;
b) conjugating the oligonucleotide of claim 3 to the DNA of step a);
c) incubating said duplex DNA under conditions which denature said duplex DNA, thereby producing single stranded DNA;
d) contacting the single stranded DNA of step d) with at least one deaminase;
e) PCR amplifying the deaminase treated DNA; and
f) sequencing PCR amplicons obtained from step e) and generating methylation profiles for said target DNA molecule.

18. The method of claim 17, wherein the DNA of step a) or step b) is treated with at least one of glucosyltransferase, methyltransferase, polymerase, and/or TET enzyme, and the appropriate substrate therefor, a chemical agent for deamination selected from bisulfite, pyridine borane, or borane-mediated deamination reagent;
or
wherein the DNA of step a) or step b) is contacted with a glucosyltransferase and a UDP glucose derivative, thereby site specifically labeling all 5hmC bases with a glucose or modified glucose prior to performance of steps b)-g);
or
wherein said DNA in step a) or b) is contacted with at least one TET enzyme thereby catalyzing oxidation of 5mC to 5hmC, 5hmC to 5fC and 5fC to 5caC prior to performance of downstream steps
or
wherein said DNA in step a) or b) is contacted with a methyltransferase, thereby converting unmodified cytosines in the methyltransferase recognition sites of said DNA into 5-modified-cytosines.

19.-23. (canceled)

24. The method of claim 17, wherein said DNA in step b) is copied by a polymerase with unmodified or non-deamination-resistant dCTP analogs to generate a copy strand of the target DNA that contains deamination-susceptible cytosines, or
wherein said DNA in step b) is copied by a polymerase with deamination-resistant dCTP analogs to generate a copy strand of the target DNA that contains deamination-resistant cytosines;
or
wherein said DNA in step b) is copied by a polymerase with deamination-resistant dCTP analogs in a copy strand of the target DNA that contains deamination-resistant cytosines, and wherein the two strands of an original DNA strand and copy DNA strand are conjugated via an oligonucleotide adapter, which can be the same or different from the adapter of step b).

25.-26. (canceled)

27. A method for reiterative assessment of the methylation state of the same DNA molecule in library constructs, comprising;
a) providing a nucleic acid sample comprising methylated DNA wherein said DNA is optionally sheared or is naturally 500 to 1000 nucleotides in length;
b) ligating the oligonucleotide of claim 3 to the DNA of step a), optionally containing a unique barcode sequence in the oligonucleotide;
c) immobilization and deamination of the DNA sample with steps i), ii), and iii) performed any operable order;
i) contacting the DNA of step b) with a solid support comprising the second member of said specific binding pair, thereby forming a duplex DNA containing specific binding member pair complex on a surface of said solid support;
ii) treating duplex DNA with bisulfite, thereby converting cytosine to uracil and converting 5hmC to adduct CMS;
iii) amplifying and sequencing the bisulfite-treated DNA thereby creating a first library of constructs comprising a first set of barcode, for identifying 5mC and 5hmC present in said sequence;
and
iv) treating said duplex DNA containing specific binding pair complex of step c) with enzymatic deamination, thereby converting residual 5mC to T, and thereby creating a second library of constructs comprising a second set of barcodes, for identifying 5hmC present in said sequence;
d) comparing said first and second sets of barcodes present in the first and second library constructs, thereby identifying 5mC and 5hmC modifications present in the original starting molecule of step a).

28. The method of claim 27, where the DNA of step a), b) or step c) is treated with at least one glucosyltransferase, methyltransferase, and TET enzyme, and the appropriate substrate therefor,
or
wherein said DNA in step a), b) or step c) is contacted with a glucosyltransferase and a UDP glucose derivative, thereby site specifically labeling all 5hmC bases glucose or a modified glucose prior to performance of downstream steps,
or
wherein said DNA in step a), b) or step c) is contacted with at least one TET enzyme thereby catalyzing oxidation of 5mC to 5hmC, 5hmC to 5fC and 5fC to 5caC prior to performance of downstream steps
or
wherein said DNA in step a), b) or c) is contacted with a methyltransferase, thereby converting unmodified cytosines in the methyltransferase recognition sites of said DNA into 5-modified-cytosines.

29.-32. (canceled)

33. The method of claim 27, wherein said DNA in step b) or c) is copied by a polymerase with unmodified or non-deamination-resistant dCTP analogs to generate a copy strand of the target DNA that contains chemical/enzymatic deamination-susceptible cytosines;
or
wherein said DNA in step b) or c) is copied by a polymerase with deamination-resistant dCTP analogs to generate a copy strand of the target DNA that contains chemical/enzymatic deamination-resistant cytosines;
or
wherein said DNA in step b) or c) is copied by a polymerase with deamination-resistant dCTP analogs n a copy strand of the target DNA that contains deamination-resistant cytosines, and wherein the two strands of an original DNA strand and copy DNA strand are conjugated via an oligonucleotide adapter, which can be the same or different from the adapter of step b).

34.-35. (canceled)

36. The method of claim 1, wherein said DNA is obtained from tissue, tumor cell, blood, plasma, serum, urine, effusion cerebrospinal fluid, lavage, breast milk, synovial fluid, saliva, sputum, tears, abscess, aspirate, swab, and nasal secretion.

37. The method of claim 1, wherein said DNA is circulating cell free DNA (cfDNA) present in one or more of serum, plasma, diseased tissue or of fetal origin in maternal circulation.

38.-39. (canceled)

40. A kit comprising comprising an oligonucleotide as claimed in claim 1 operably linked to a first member of a specific binding pair, wherein said adapter renders the oligonucleotide resistant to deamination, a solid support operably linked to a second member of the specific binding pair, which when incubated together forms a DNA containing binding complex, deamination enzymes, and optionally one or more of a polymerase enzyme, a helicase enzyme, a glucosyl transferase enzyme, a TET enzyme, a methyltransferase enzyme and the appropriate substrates thereof.

41.-42. (canceled)
